(19) United States
(12) Patent Application Publication
(10) Pub. No.: US 2022/0333192 A1
(43) Pub. Date: Oct. 20, 2022

(54) METHODS AND DEVICES FOR SPATIAL ASSESSMENT OF RNA QUALITY

(71) Applicant: 10x Genomics, Inc., Pleasanton, CA (US)

(72) Inventor: Cedric Uytingco, Pleasanton, CA (US)

(21) Appl. No.: 17/724,977

(22) Filed: Apr. 20, 2022

Related U.S. Application Data
(60) Provisional application No. 63/177,165, filed on Apr. 20, 2021.

(51) Int. Cl. C12Q 1/6874

(52) U.S. Cl. CPC C12Q 1/6874; C12Q 1/6818

(57) ABSTRACT

Provided herein are methods and kits for determining nucleic acid integrity of a biological sample. Disclosed are methods comprising: (a) contacting a biological sample with a substrate comprising a plurality of capture probes; (b) hybridizing a nucleic acid analyte from the biological sample to the capture probe; (c) extending a 3′ end of the capture probe using the nucleic acid analyte as a template to generate an extended capture probe; (d) hybridizing a plurality of labeled oligonucleotide probes comprising a first label and a second label to the extended capture probe; and (e) detecting a first intensity of the first label and a second intensity of the second label of the plurality of labeled oligonucleotides hybridized to the extended capture probe, thereby detecting a length of the nucleic acid analyte and determining the nucleic acid integrity of the biological sample.

### CROSS-REFERENCE TO RELATED APPLICATION

This application claims priority to U.S. Provisional Patent Application No. 63/177,165, filed Apr. 20, 2021. The content of this application is incorporated herein by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

The integrity of biological samples used in the study of spatial heterogeneity is critical to obtaining optimal data that leads to the best understanding of gene expression of the biological samples. Methods of assessing RNA integrity offer the determination of overall RNA quality in a biological sample. However, these methods are restricted to homogenizing the biological samples, thereby losing spatial representation. Thus, there remains a need to develop a quality assessment tool that is able to assess RNA integrity within a spatial context of a biological sample.

### SUMMARY

Determining the RNA integrity of an analyte in a biological sample by measuring analyte length and correlating the measured length with the spatial location of the analyte in a sample offers an attractive alternative to RNA integrity assessment methods where a biological sample is homogenized. The methods of determining RNA integrity disclosed herein provide a mapping of RNA integrity of the biological sample with respect to spatial locations on the biological sample. In this manner, the methods disclosed herein provide a user with information regarding not only the overall viability of a biological sample but additionally, with location-specific RNA integrity values.

In one aspect, this disclosure includes methods of determining nucleic acid (e.g., RNA) integrity of a biological sample. The method includes: (a) contacting the biological sample with a substrate comprising a plurality of capture probes, wherein a capture probe of the plurality of capture probes comprises a capture domain; (b) hybridizing a nucleic acid analyte of the biological sample to the capture probe; (c) extending a 3′ end of the capture probe using the nucleic acid analyte as a template to generate an extended capture probe; (d) hybridizing a plurality of labeled oligonucleotide probes to the extended capture probe, wherein the plurality of labeled oligonucleotide probes includes: (i) a primer oligonucleotide probe comprising a first label, and (ii) a plurality of labeled oligonucleotide probes comprising one or more copies of a second label, wherein the plurality of labeled oligonucleotide probes comprising the second label are substantially complementary to the extended capture probe, or a complement thereof; (e) detecting a first intensity of the first label and a second intensity of the second label of the plurality of labeled oligonucleotides hybridized to the extended capture probe, thereby detecting a length of the nucleic acid analyte; and (f) calculating a ratio of the second intensity to the first intensity, thereby determining the nucleic acid (e.g., RNA) integrity of the biological sample.

In some embodiments, the first label comprises a first fluorescent label. In some embodiments, the second label comprises a second fluorescent label that is different from the first fluorescent label. In some embodiments, the methods further include comparing the ratio to one or more references, wherein each of the one or more references comprises a labeled oligonucleotide having a known length and a known intensity. In some embodiments, the first intensity is indicative of a capture of the nucleic acid analyte by the capture probe. In some embodiments, the second intensity is indicative of the length of the nucleic acid analyte. In some embodiments, the primer oligonucleotide probe has a length that ranges from about 50 nucleotides to about 100 nucleotides. In some embodiments, each labeled oligonucleotide probe comprising the second label has a length that ranges from about 50 nucleotides to about 100 nucleotides. In some embodiments, each labeled oligonucleotide probe comprising the second label of the plurality of labeled oligonucleotide probes has a length that is about 5% to about 20% of a length of the extended capture probe. In some embodiments, the nucleic acid (e.g., RNA) integrity is determined to be high when the ratio of the second intensity to the first intensity is about 3:1 to about 10:1.

In some embodiments, the method further comprises generating an image of the extended capture probe and using the image of the extended capture probe to generate a spatial nucleic acid integrity number for a location on the substrate. In some embodiments, the nucleic acid analyte is a ribosomal RNA (rRNA) or a transfer RNA (tRNA). In some embodiments, the rRNA is 18S rRNA, 28S rRNA, or a combination thereof. In some embodiments, if the nucleic acid integrity is determined to be high, the method further comprises determining abundance and location of a plurality of analytes in a related biological sample, wherein the related biological sample is a serial tissue section from the biological sample. In some embodiments, the method further comprises imaging the serial tissue section.

In some embodiments, the determining the abundance and location comprises: (a) contacting a spatial array comprising a comprising a plurality of spatial capture probes with the serial tissue section, wherein a spatial capture probe of the plurality of spatial capture probes comprises a spatial barcode and a spatial capture domain; (b) hybridizing an analyte from the plurality of analytes within the serial tissue section to a spatial capture domain; and (c) determining (i) all or a part of a sequence of the analyte from the serial tissue section, or a complement thereof, and (ii) the spatial barcode, or a complement thereof, and using the determined sequence of (i) and (ii) to determine the abundance and the location of the analyte from the serial tissue section.

In some embodiments, the determining in step (f) comprises amplifying all or part of the analyte hybridized to the capture domain, or a complement thereof, wherein the amplifying creates an amplification product comprising: (i) all or part of the analyte hybridized to the capture domain, or a complement thereof, and (ii) the spatial barcode, or a complement thereof. In some embodiments, the determining in step (f) comprises sequencing. In some embodiments, the spatial capture probe further comprises one or more functional domains, a unique molecular identifier, a cleavage domain, or a combination thereof. In some embodiments, an increase in the length of the nucleic acid analyte compared to a reference of the one or more references correlates to an increase of the nucleic acid integrity of the biological sample.

In some embodiments, the nucleic acid integrity is RNA integrity. In some embodiments, the biological sample is a tissue sample, wherein the tissue sample is a fresh frozen tissue sample or a fixed tissue sample. In some embodiments, the capture domain comprises a sequence complementary to the nucleic acid analyte, wherein the nucleic acid analyte hybridizes to the capture probe via the capture domain.

In another aspect, this disclosure includes methods of determining RNA integrity of a biological sample. The methods include: (a) contacting the biological sample with a substrate comprising a plurality of capture probes, wherein a capture probe of the plurality of capture probes comprises a capture domain; (b) hybridizing an RNA analyte from the biological sample to the capture probe; (c) extending a 3′ end of the capture probe using the RNA analyte as a template to generate an extended capture probe; (d) hybridizing a first labeled oligonucleotide probe and a second labeled oligonucleotide probe to the extended capture probe, wherein the first labeled oligonucleotide probe is capable of transferring energy to the second labeled oligonucleotide probe by a dipole-dipole coupling mechanism; and (e) determining the energy transferred to the second labeled oligonucleotide probe, thereby detecting a length of the RNA analyte and determining the RNA integrity of the biological sample.

In some embodiments, the dipole-dipole coupling mechanism is Forster resonance energy transfer (FRET). In some embodiments, the first labeled oligonucleotide probe and the second labeled oligonucleotide probe are hybridized in an alternating pattern to the extended capture probe. In some embodiments, the first labeled oligonucleotide probe and the second labeled oligonucleotide probe have a length that ranges from about 50 nucleotides to about 100 nucleotides. In some embodiments, the first labeled oligonucleotide probe and the second labeled oligonucleotide probe have a length that is about 1% to about 10% of a length of the extended capture probe. In some embodiments, the methods further include measuring a FRET efficiency of the first and second labeled oligonucleotide probes. In some embodiments, the RNA integrity is high when the FRET efficiency ranges from about 70% to about 100%. In some embodiments, the methods further include generating an image of the extended capture probe and using the image of the extended capture probe to generate a spatial RNA integrity number for a location on the substrate. In some embodiments, the RNA integrity is high when the length of the RNA analyte is about 50% to about 90% of an intact length of the RNA analyte. In some embodiments, the RNA integrity is high when the length of the RNA analyte is about 900 to about 1600 nucleotides. In some embodiments, the RNA integrity is high when the length of the RNA analyte is about 2500 to about 4500 nucleotides. In some embodiments, an increase in the length of the RNA analyte is correlated to an increase of the RNA integrity of the biological sample.

In some embodiments of the disclosed methods, the biological sample is a tissue sample. In some embodiments, the tissue sample comprises a tissue section, a region within a tissue, or a single cell within a tissue. In some embodiments, the capture domain comprises a sequence complementary to the nucleic acid analyte (e.g., RNA). In some embodiments, the capture domain comprises a poly(T) sequence. In some embodiments, the nucleic acid analyte is a ribosomal RNA (rRNA). In some embodiments, the rRNA is 18S rRNA, 28S rRNA, or a combination thereof. In some embodiments, the methods further include calculating a ratio of the length of the 18S rRNA and/or the 28S rRNA. In some embodiments, the RNA integrity is high when the ratio of the 18S rRNA and/or the 28S rRNA is greater than or equal to about 2. In some embodiments, the nucleic acid analyte is a transfer RNA (tRNA). In some embodiments, the analyte is a highly-expressed analyte.

In some embodiments, if the nucleic acid (e.g., RNA) integrity is high, the method further comprises determining abundance and location of a plurality of analytes in a related biological sample, wherein the related biological sample is a serial tissue section from the biological sample. In some embodiments, determining the abundance and location includes: contacting a spatial array comprising a comprising a plurality of spatial capture probes with the serial tissue section, wherein a spatial capture probe of the plurality of spatial capture probes comprises a spatial barcode and a spatial capture domain; hybridizing an analyte from the serial tissue section to a spatial capture domain; and determining (i) all or a part of a sequence of the analyte from the serial tissue section, or a complement thereof, and (ii) the spatial barcode, or a complement thereof, and using the determined sequence of (i) and (ii) to determine the abundance and the location of the analyte from the serial tissue section.

In some embodiments, the determining step comprises amplifying all or part of the analyte from the serial tissue section hybridized to the capture domain, or a complement thereof, wherein the amplifying creates an amplification product comprising: (i) all or part of the analyte from the serial tissue section bound to the capture domain, or a complement thereof, and (ii) all or a part of the sequence of the spatial barcode, or a complement thereof. In some embodiments, the determining step comprises sequencing. In some embodiments, the methods further include imaging the serial tissue section. In some embodiments, the spatial capture probe further comprises one or more functional domains, a unique molecular identifier, a cleavage domain, or a combination thereof.

In yet another aspect, the disclosure provides substrates including: one or more discrete sample regions configured to receive a biological sample; a capture probe attached to a location on the sample region, the capture probe configured to hybridize a sample analyte of the biological sample; and one or more discrete reservoirs defined by a surface of the substrate and linearly arranged on the substrate, the one or more reservoirs defining a volume configured to receive a fluorescently-labeled reference analyte having a known length.

In some embodiments, the fluorescently-labeled reference analyte is 18S rRNA, 28S rRNA, or a combination thereof. In some embodiments, the known length ranges from about 10 to about 1800 nucleotides. In some embodiments, the known length ranges from about 10 to about 5000 nucleotides. In some embodiments, the volume ranges from about 20 μL to about −700 μL.

In some embodiments, the substrate further comprises an array disposed on a surface of the substrate, the array comprising a reference analyte having a known length. In some embodiments, the methods further include a reference analyte having a known length.

In another aspect, the disclosure provides kits including: (a) a substrate comprising a plurality of capture probes wherein a capture probe of the plurality of capture probes comprises a capture domain; (b) a plurality of labeled oligonucleotide probes comprising: (i) a primer oligonucleotide probe comprising a first fluorescent label, and (ii) a plurality of labeled oligonucleotide probes comprising a second fluorescent label, wherein the plurality of labeled oligonucleotide probes comprising the second fluorescent label are substantially complementary to a nucleic acid analyte, or a complement thereof, and (c) instructions for performing one or more methods of the disclosure.

In another aspect, the disclosure provides kits including: (a) a substrate comprising a plurality of capture probes wherein a capture probe of the plurality of capture probes comprises a capture domain; (b) a first labeled oligonucleotide probe and a second labeled oligonucleotide probe, wherein the first labeled oligonucleotide probe is capable of transferring energy to the second labeled oligonucleotide probe by a dipole-dipole coupling mechanism, and wherein the first labeled oligonucleotide comprises a donor fluorophore, and the second labeled oligonucleotide comprises an acceptor fluorophore; and (c) instructions for performing one or more methods of the disclosure.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, where the cleaved capture probe can enter a non-permeabilized cell and bind to target analytes within the cell.

FIG. 3 is a schematic diagram of an example multiplexed spatially-barcoded feature.

FIG. 4 is a schematic diagram of an example analyte capture agent.

FIG. 5 is a schematic diagram depicting an example interaction between a feature-immobilized capture probe and an analyte capture agent.

FIGS. 6A-6C are schematics illustrating how streptavidin cell tags can be utilized in an array-based system to produce spatially-barcoded cells or cellular contents.

FIG. 7 shows a schematic diagram and flowchart depicting an example RNA integrity determination process using a plurality of labeled oligonucleotide probes.

FIGS. 8A-8B are schematics illustrating a plurality of example labeled oligonucleotide probes hybridized to capture probes of different lengths.

FIG. 9 is a schematic illustrating an example visual representation of the quantification of RNA integrity via ratiometric imaging.

FIG. 10 shows a schematic diagram and flowchart depicting an example RNA integrity determination process using first and second labeled oligonucleotide probes.

FIGS. 11A-11C are schematics illustrating example first and second labeled oligonucleotide probes hybridized to capture probes of different lengths.

FIGS. 12A-12B are schematics illustrating example visual representations of the quantification of RNA integrity via Forster resonance energy transfer (FRET) efficiency.

FIGS. 13A-13C are schematics illustrating example substrates including reservoirs.

FIGS. 14A-14B are schematics illustrating example substrates including arrays.

### DETAILED DESCRIPTION

### I. Introduction

The integrity of RNA is an important quality to measure or estimate when selecting samples (e.g., biological samples) for downstream analysis (e.g., RNA and/or DNA sequencing). For next generation sequencing (NGS), it is critical to use biological samples that have the highest amount of intact messenger RNA (mRNA) (e.g., transcript). Fragmented or degraded mRNA can lead to a poor understanding of gene expression of the biological sample and a waste of resources. Furthermore, in spatial NGS assays, certain regions of the sample (e.g., a tissue sample) may exhibit ideal or poor mRNA quality due in part to poor sample preparation or disease where there exists regional variation. Therefore, there is a need for a quality assessment of the integrity of RNA at the level of sample preparation to ensure that the mRNA or other analytes are intact prior to running an assay (e.g., RNA and/or DNA sequencing).

Determining the RNA integrity of an analyte in a biological sample by measuring analyte length and correlating the measured length with the spatial location of the analyte in a sample offers an attractive alternative to RNA integrity assessment methods where a biological sample is homogenized. The devices and methods of determining RNA integrity disclosed herein provide a mapping of RNA integrity of the biological sample with respect to spatial locations on the biological sample.

Some embodiments of the devices and methods described herein may provide one or more of the following advantages. In some embodiments, the methods and devices of the disclosure are advantageously not restricted to homogenizing the samples and achieve in situ spatial representation of RNA integrity at the cellular level. In some embodiments, the methods and devices disclosed herein can provide a user with information regarding the overall viability of a biological sample. In some embodiments, the methods and devices disclosed herein can provide a user with location-specific RNA integrity values. In some embodiments, the methods and devices disclosed herein can provide a user with data visualization (e.g., a heat map) indicating the in situ spatial variation in tissue quality of a sample. In some embodiments, the methods and devices disclosed herein facilitate a more efficient workflow (e.g., a NGS workflow) as it does not require lengthy steps, such as the use of gel electrophoresis to determine the degree of fragmentation of a desired analyte and/or the extraction of total RNA from the biological sample, to assess the quality and/or viability of a biological sample. In some embodiments, the methods and devices disclosed allow for the assessment and determination of superior sample handling and preparation methods for a spatial workflow. In some embodiments, the methods and devices help distinguish between decreases in RNA integrity due to, for example, sample handling compared to decreases in RNA integrity as a result of biological activity, for example, tissue necrosis due to myocardial infarction, etc.

The present disclosure examines the spatial RNA Integrity Number in a biological sample. As used herein, the term “spatial RNA Integrity Number” or “sRIN” refers to the in situ indication of RNA quality based on an integrity score. Higher sRIN scores correlate with higher data quality in the spatial profiling assays described herein. For example, a first biological sample with a high sRIN score will have higher data quality compared to a second biological sample with sRIN score lower than the first biological sample. In some embodiments, a sRIN is calculated for a tissue section, one or more regions of a tissue section, or a single cell.

In some embodiments, one or more sRINs for a given biological sample (e.g., tissue section, one or more regions of a tissue, or a single cell) are calculated by: (a) providing (i) a spatial array including a plurality of capture probes on a substrate, where a capture probe comprises a capture domain and (ii) a tissue stained with a histology stain (e.g., any of the stains described herein); (b) contacting the spatial array with the biological sample (e.g., tissue); (c) capturing a biological analyte (e.g., an 18S rRNA molecule) from the biological sample (e.g., tissue) with the capture domain; (d) generating a cDNA molecule from the captured biological analyte (e.g., 18S rRNA); (e) hybridizing one or more labeled oligonucleotide probes to the cDNA; (f) imaging the labeled cDNA and the histology stain (e.g., any of the stains described herein), and (g) generating a spatial RNA integrity number for a location in the spatial array, wherein the spatial RNA integrity number comprises an analysis of a labeled cDNA image and a histology stain (e.g., any of the stains described herein) image for the location.

In some embodiments, the biological sample (e.g., tissue) is stained with a histology stain. As used herein, a “histology stain” can be any stain described herein. For example, the biological sample can be stained with IF/IHC stains described herein. For example, the biological sample (e.g., tissue) can be stained with Hematoxylin & Eosin (“H&E”). In some embodiments, the biological sample (e.g., tissue) is stained with a histology stain (e.g., any of the stains described herein) before, contemporaneously with, of after labelling of the cDNA with labeled oligonucleotide probes. In some embodiments, the stained biological sample can be, optionally, de-stained (e.g., washed in HCl). For example, Hematoxylin, from the H&E stain, can be optionally removed from the biological sample by washing in dilute HCl (0.01M) prior to further processing. In some embodiments, the stained biological sample can be optionally de-stained after imaging and prior to permeabilization.

In some embodiments, the spatial array includes a plurality of capture probes immobilized on a substrate where the capture probes include at least a capture domain. In some embodiments, the capture domain includes a poly(T) sequence. For example, a capture domain includes a poly(T) sequence that is capable of capturing an 18S rRNA transcript from a biological sample.

In some embodiments, calculating one or more spatial RNA Integrity Numbers for a biological sample includes hybridizing at least one (e.g., at least two, at least three, at least four, or at least five) labeled oligonucleotide probes to the cDNA generated from the 18s rRNA. In some embodiments, a labeled oligonucleotide probe includes a sequence that is complementary to a portion of the 18S cDNA. In some embodiments, four labeled oligonucleotide probes (P1-P4) are designed to hybridize at four different locations spanning the entire gene body of the 18S rRNA. In some embodiments, a labeled oligonucleotide probe can include any of the detectable labels as described herein. For example, an oligonucleotide labeled probe can include a fluorescent label (e.g., Cy3, ATTO550, ATT0594, Alexa Fluor™ 594). In some embodiments, one or more of the labeled oligonucleotide probes designed with complementarity to different locations within the 18S cDNA sequence include the same detectable label. For example, four labeled oligonucleotide probes, (P1-P4) each designed to have complementarity to a different location within the 18S cDNA sequence can all have the same detectable label (e.g., Cy3, ATTO550, ATT0594, Alexa Fluor™ 594). In some embodiments, one or more of the labeled oligonucleotide probes designed with complementarity to different locations within the 18S cDNA sequence include a different detectable label. For example, four labeled oligonucleotide probes, (P1-P4) each designed to have complementarity to a different location within the 18S cDNA sequence can include different detectable labels.

In some embodiments, determining a spatial RNA Integrity Number for a biological sample (e.g., tissue section, one or more regions of a tissue, or a single cell) includes analyzing the images taken from a spatial array and a histology stain (e.g., any of the stains described herein) for the same location. For example, for the spatial array, all images are generated by scanning with a laser (e.g., a 532 nm wavelength) after the fluorescently labeled (e.g., Cy3) oligonucleotide probes have been hybridized to the 18S cDNA. One image is generated per probe (P1-P4) and one image is generated where no fluorescently labeled probes were hybridized (P0). Normalization of Fluorescence Units (FU) data is performed by subtraction of the auto-fluorescence recorded with P0 and division with P1. After alignment, the five images (one image from each probe, P1-P4, and one image from an area without bound probe) are loaded into a script. The script generates two different plots, one heat-map of spatial RIN values and one image alignment error plot, which combines the histology stain (e.g., any of the stains described herein) image. The image alignment error plot is used to visualize which pixels and positions should be excluded from the analysis due to alignment errors between the images from P0-P4.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein. Examples of nucleic acid analytes include, but are not limited to, DNA (e.g., genomic DNA, cDNA) and RNA, including coding and non-coding RNA (e.g., mRNA, rRNA, tRNA, ncRNA).

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be an ex vivo sample, a three-dimensional (3D) cell culture sample, an organoid, and/or any other heterogenous cell cultures. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Example permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

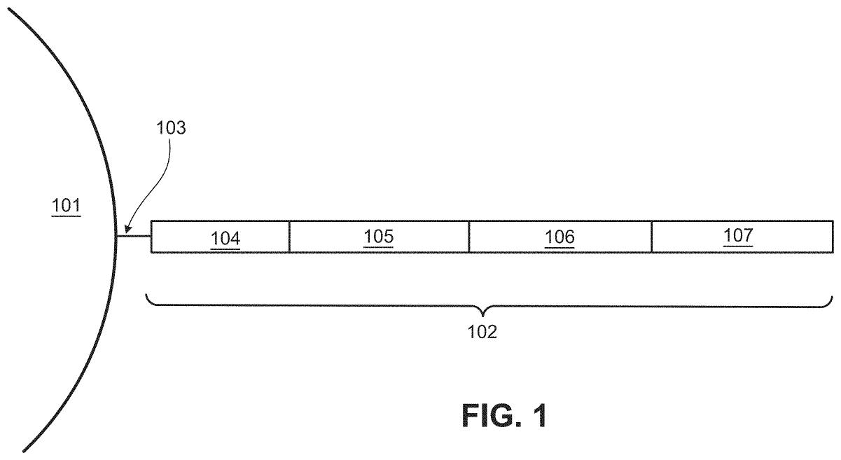

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107 or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

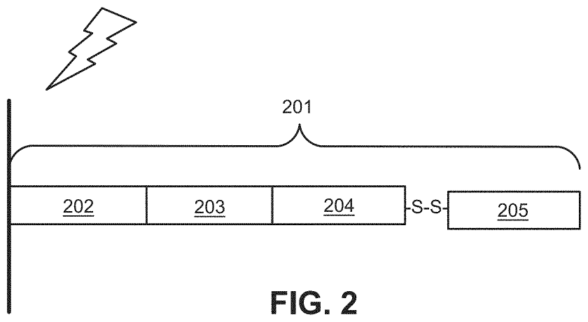

FIG. 2 is a schematic illustrating a cleavable capture probe, wherein the cleaved capture probe can enter a non-permeabilized cell and bind to analytes within the cell. The capture probe 201 contains a cleavage domain 202, a cell penetrating peptide 203, a reporter molecule 204, and a disulfide bond (—S—S—). 205 represents all other parts of a capture probe, for example a spatial barcode, a UMI and a capture domain.

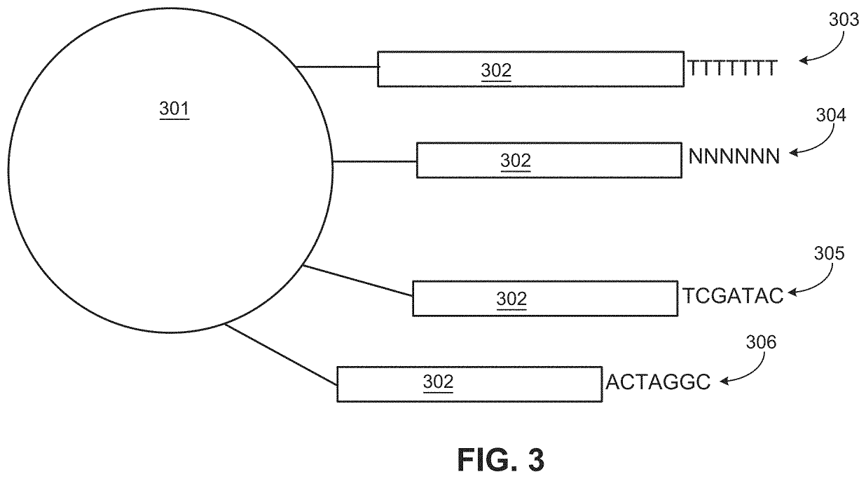

FIG. 3 is a schematic diagram of an exemplary multiplexed spatially-barcoded feature. In FIG. 3, the feature 301 can be coupled to spatially-barcoded capture probes, where the spatially-barcoded probes of a particular feature can possess the same spatial barcode but have different capture domains designed to associate the spatial barcode of the feature with more than one target analyte. For example, a feature may be coupled to four different types of spatially-barcoded capture probes, each type of spatially-barcoded capture probe possessing the spatial barcode 302 and a different capture domain. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the scheme shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

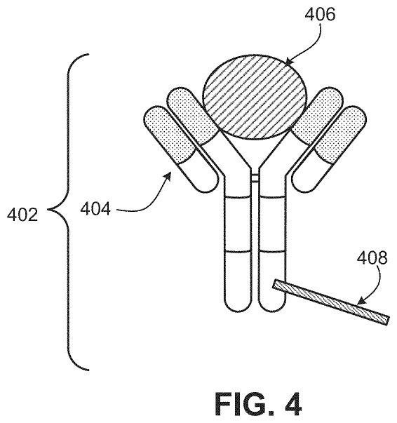

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

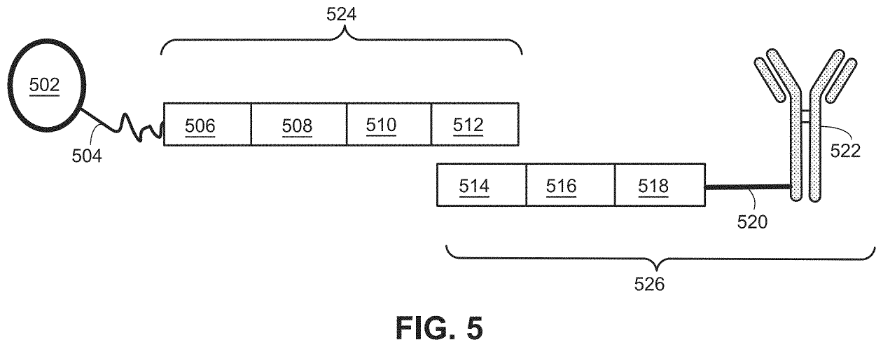

FIG. 5 is a schematic diagram depicting an exemplary interaction between a capture probe 524 immobilized on a feature 502 via a linker 504 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

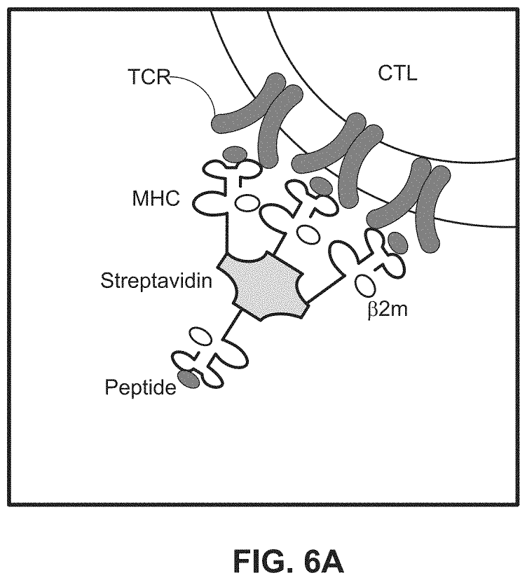

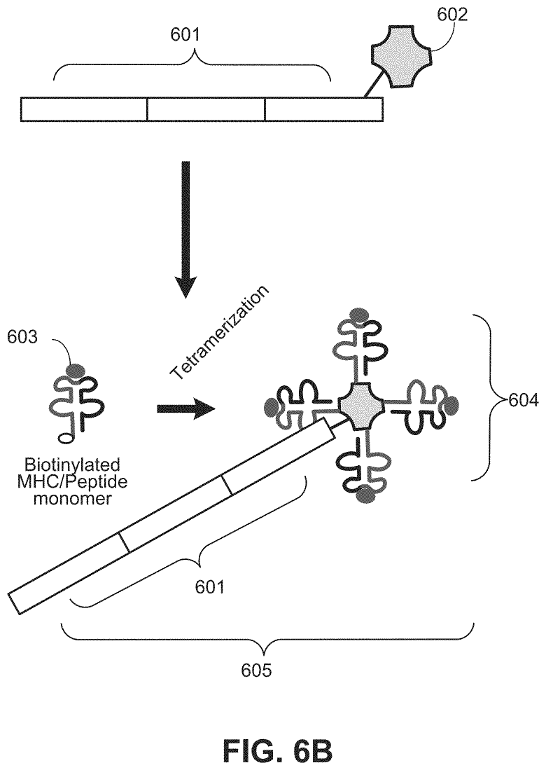

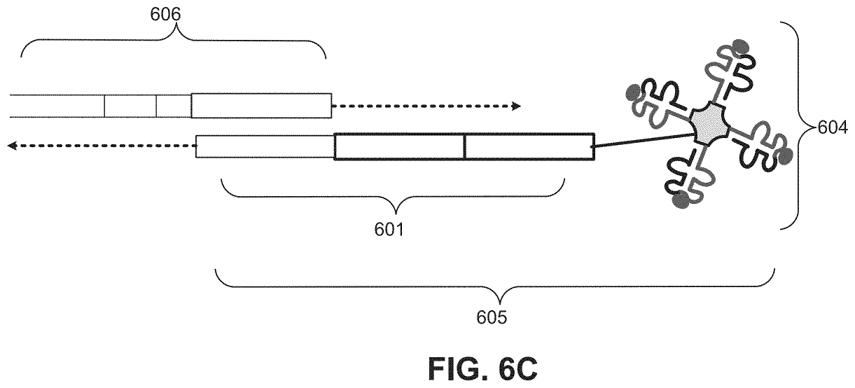

FIGS. 6A, 6B, and 6C are schematics illustrating how streptavidin cell tags can be utilized in an array-based system to produce a spatially-barcoded cell. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a T-Cell Receptor (TCR) such that the streptavidin binds to a target T-cell via multiple MCH/TCR binding interactions. Multiple interactions synergize and can substantially improve binding affinity. Such improved affinity can improve tagging of T-cells and also reduce the likelihood that tags will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated tag and also includes optional functional sequences such as sequences for hybridization with other oligonucleotides. As shown in FIG. 6C, one exemplary oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from the spatial barcode sequence on the capture agent barcode domain 601 may be used to identify the particular peptide MHC complex 604 bound to the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells (e.g., in a tissue sample), such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto cells of the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods of Determining RNA Integrity

(a) Background

Disclosed herein, in certain embodiments, are methods directed to determining RNA integrity of a biological sample (e.g., a tissue sample). In some embodiments, the methods include contacting the biological sample with a substrate comprising a plurality of capture probes. In some embodiments, a capture probe of the plurality of capture probes comprises a capture domain. In some embodiments, the methods include hybridizing an analyte (e.g., a nucleic acid analyte) to the capture probe (e.g., to the capture domain of the capture probe). In some embodiments, the methods include extending a 3′ end of the capture probe using the analyte as a template to generate an extended capture probe. In some embodiments, the methods include hybridizing a plurality of labeled oligonucleotide probes to the extended capture probe. The RNA integrity of a sample can be defined based on the relative abundance of full-length analytes (e.g., RNA transcripts). High-quality samples may comprise predominantly full-length transcripts while low-quality samples may comprise mostly short, fragmented transcripts. As such, the methods of the disclosure can determine the RNA integrity and quality of a biological sample by detecting a signal generated by the labeled oligonucleotide probes. For example, in some embodiments, the methods can include measuring and/or imaging a fluorescence intensity of the plurality of labeled oligonucleotide probes and determining the RNA integrity by correlating fluorescence intensity values to lengths of the plurality of labeled oligonucleotide probes. Additionally, in certain embodiments, the methods can include measuring the amount of energy transferred between a first and second labeled oligonucleotide probe and determining the RNA integrity of a sample based on an energy transfer efficiency. Both of these methods are discussed in detail below.

(b) Relative Fluorescent Signal Methods

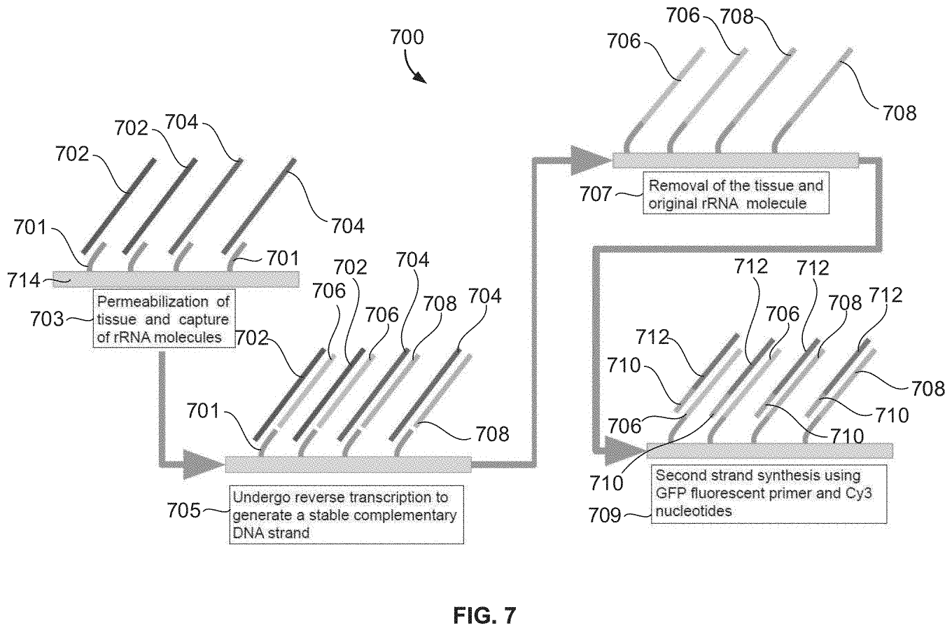

In some embodiments, the methods disclosed herein are directed to capturing an analyte of a biological sample on the surface of a substrate and determining a length of the analyte. FIGS. 7-9 illustrate methods to determine the length of the analyte via detection of a fluorescence intensity signal of one or more incorporated fluorescent nucleotides. In some embodiments, the samples or regions of a sample exhibiting a lower fluorescent signal ratio (relative to a reference) can be considered to have lower RNA integrity. In the same manner, the samples or regions of a sample exhibiting a higher fluorescent signal ratio (relative to a reference) can be considered to have higher RNA integrity.

Referring to FIG. 7, a method (700) of determining RNA integrity of a biological sample (e.g., a tissue sample) initially involves contacting the biological sample with a substrate 714 comprising a plurality of capture probes 701. In some embodiments, the biological sample comprises a tissue section, a region within a tissue, or a single cell within a tissue. In some embodiments, the capture probes 701 are designed to hybridize to one or more desired analytes of the biological sample. For example, as shown in FIG. 7, the capture probes 701 can be designed to hybridize to a first analyte 702 and a second analyte 704. In some embodiments, the first analyte 702 and/or the second analyte 704 are nucleic acid molecules (e.g., an RNA molecule). In some embodiments, the first analyte 702 and/or the second analyte 704 are ribosomal RNA (rRNA) molecules, messenger RNA (mRNA) molecules, transfer RNA (tRNA) molecules, or a combination thereof. In some embodiments, the first analyte 702 is an 18S ribosomal RNA (rRNA) molecule. In some embodiments, the second analyte 704 is a 28S rRNA molecule. In some embodiments, the first analyte 702 and/or the second analyte 704 are analytes that are expressed in high quantities in the biological sample. In some embodiments, the concentration of the first analyte 702 or the second analyte 704 in a biological sample ranges from at least about 20 ng/μL to about 400 ng/μL (e.g., from about 20 ng/μL to about 30 ng/μL, from about 30 ng/μL to about 40 ng/μL, from about 40 ng/μL to about 50 ng/μL, from about 50 ng/μL to about 60 ng/μL, from about 60 ng/μL to about 70 ng/μL, from about 70 ng/μL to about 80 ng/μL, from about 80 ng/μL to about 90 ng/μL, from about 90 ng/μL to about 100 ng/μL, from about 100 ng/μL to about 150 ng/μL, from about 150 ng/μL to about 200 ng/μL, from about 200 ng/μL to about 250 ng/μL, from about 250 ng/μL to about 300 ng/μL, from about 300 ng/μL to about 350 ng/μL, from about 350 ng/μL to about 400 ng/μL). In some embodiments, the first analyte 702 and/or the second analyte 704 are overexpressed or highly-expressed analytes in a biological sample containing one or more cancerous cells. In some embodiments, the first analyte 702 and/or the second analyte 704 are underexpressed analytes in a biological sample. For example, in some embodiments, the overexpressed, highly-expressed, or underexpressed analytes include, but are not limited to, transfer RNA (tRNA) (e.g., nuclear-encoded tRNA and/or mitochondrial-encoded tRNA), a microRNA, and a small nuclear RNA (snRNA). In some instances, the overexpressed or highly-expressed analyte is a housekeeping gene and/or an RNA transcript (e.g., mRNA) of a housekeeping gene. Housekeeping genes include, but are not limited, to 18S rRNA, 28S rRNA, glyceraldehyde-3-phosphate dehydrogenase (GAPDH), microtubule affinity regulating kinase 3 (MARK3), b2 microglobulin (B2M), beta-actin (ACTB), phosphoglycerate kinase 1 (PGK1), peptidylprolyl isomerase A (PPIA), ribosomal protein L13a (RPL13A), ribosomal protein, large, P0 (RPLP0), acidic ribosomal phosphoprotein PO (ARBP), asparagine-linked glycosylation 9 (ALG9), ribosomal protein L13a (RPL13A), hydroxymethylbilane synthase (HMBS), hypoxanthine phosphoribosyltransferase 1 (HPRT1), and succinate dehydrogenase (SDPH). In some embodiments, the housekeeping gene is human acidic ribosomal protein (HuPO), j-actin (BA), cyclophilin (CYC), glyceraldehyde-3-phosphate dehydrogenase (GAPDH), phosphoglycerokinase (PGK), β₂-microglobulin (B2M), β-glucuronidase (GUS), hypoxanthine phosphoribosyltransferase (HPRT), transcription factor IID TATA binding protein (TBP), transferrin receptor (TfR), elongation factor-1-α (EF-1-α), metastatic lymph node 51 (MLN51), ubiquitin conjugating enzyme (UbcH5B), or any combination thereof. Additional disclosure of housekeeping genes are described in Caracausi et al., Molecular Medicine Reports, (2017) 16: 2397-2410; Dundas et al., Theory Biosci. (2012) 131:215-223; Eisenberg, Trends in Gen. (2014) 30(3):119-20; and Dheda et al., Biotechniques (2018) 37(1):112-119, each of which is incorporated by reference in its entirety. In some embodiments, the overexpressed or highly-expressed analyte is 18S rRNA and/or 28S rRNA. In some instances, the overexpressed or highly-expressed analyte is 18S rRNA (e.g., NCBI Ref. Seq.: NR_003286.4). In some instances, the overexpressed or highly-expressed analyte is 28S rRNA (e.g., NCBI Ref. Seq.: NR_003287.4).

In some embodiments, the biological sample is permeabilized while on the substrate 714 (e.g., by providing a solution comprising a permeabilization reagent to the biological sample), and analytes from the biological sample are contacted with one or more capture probes 701 (step 703). For example, permeabilization of a biological sample can facilitate analyte release and capture. Example permeabilization agents and conditions are described in Section (I)(d)(ii)(13) or the Exemplary Embodiments Section of WO 2020/176788 and/or U.S. Patent Application Publication No. 2020/0277663. In some embodiments, one or more capture probes 701 (e.g., in an array) capture the first analyte 702 and/or the second analyte 704. In some embodiments, one or more capture probes 701 include a capture domain (e.g., a poly(T) sequence, a poly(A) sequence, a non-homopolymeric sequence). In some embodiments, the capture domain includes a sequence complementary to the first analyte 702 and/or the second analyte 704. For example, one or more capture domains can hybridize to poly(A) tails of mRNA molecules. In some instances, one or more capture domains on the array can hybridize to a portion of rRNA molecules. In some embodiments, the capture domain comprises a poly(T) sequence. In some embodiments, one or more capture probes 701 include a spatial barcode.

Reverse transcription can be carried out using a reverse transcriptase to generate a first extended capture probe 706 and a second extended capture probe 708 based on the captured, first and second analytes 702, 704, respectively (step 705). For example, in some embodiments, the methods include extending a 3′ end of the capture probe 701 using the first and/or second analytes 702, 704 as a template to generate the first and second extended capture probes 706, 708. In some embodiments, first and second extended capture probes 706, 708 are complementary DNA (cDNA) strands that are generated based on the captured, first and second analytes 702, 704, respectively. In some embodiments, reverse transcription allows for the generation of cDNA strands that stabilize the sequences on the substrate 714, thereby allowing for subsequent processing. In some embodiments, a polynucleotide tail, e.g., a poly(A) tail, is incorporated at the 3′ end of the extended probe molecules. In some embodiments, the polynucleotide tail is incorporated using a terminal transferase active enzyme. In some embodiments, the sequence and location of the captured first and second analytes 702,704 can be determined (e.g., by sequencing the capture probe that contains the spatial barcode as well as the complementary cDNA).

The biological sample including the first and second analytes 702, 704 are removed from the substrate 714 leaving behind the first and second extended capture probes 706,708 (step 707). In some embodiments, the removal step includes enzymatic and/or chemical degradation of cells of the biological sample. For example, the removal step can include treating the biological sample with an enzyme (e.g., a proteinase such as proteinase K) to remove at least a portion of the biological sample from the substrate 714. In some embodiments, the removal step includes enzymatic removal of the original analyte (e.g., rRNA, mRNA, etc.) of the biological sample. For example, the removal step can include treating the biological sample with an enzyme (e.g., an RNase). In some embodiments, the removal step can include ablation of the tissue (e.g., laser ablation).

The methods include hybridizing a plurality of labeled oligonucleotide probes to the first and/or second extended capture probes 706,708 (step 709). In some embodiments, the plurality of labeled oligonucleotide probes comprises a primer oligonucleotide probe 710 comprising a first label, and a plurality of labeled oligonucleotide probes 712 comprising a second label. In some embodiments, the first label is different from the second label. In some embodiments, the first and second labels are first and second fluorescent labels. In some embodiments, the first fluorescent label is different than the second fluorescent label. In some embodiments, the first label is green fluorescent protein (GFP). In some embodiments, the first label has an excitation maximum of about 488 nanometers (nm). In some embodiments, the first label has an emission maximum of about 510 nm. In some embodiments, the first label has an excitation range of at least about 350 nm to about 800 nm (e.g., about 350 nm to about 400 nm, about 400 nm to about 450, about 450 nm to about 500, about 500 nm to about 550, about 550 nm to about 600, about 600 nm to about 650, about 650 nm to about 700, about 700 nm to about 750, about 750 nm to about 800). In some embodiments, the second label is Cy3. In some embodiments, the second label has an excitation maximum that does not overlap, or minimally overlaps, that of the first label such as about 554 nanometers (nm). In some embodiments, the second label has an emission maximum of about 568 nm. In some embodiments, the second label has an excitation range of at least about 350 nm to about 800 nm (e.g., about 350 nm to about 400 nm, about 400 nm to about 450, about 450 nm to about 500, about 500 nm to about 550, about 550 nm to about 600, about 600 nm to about 650, about 650 nm to about 700, about 700 nm to about 750, about 750 nm to about 800). In some embodiments, the first label and the second label can be excited using a same laser line. In some embodiments, the first label and the second label can be excited using a different laser line. In some embodiments, the first label and the second label have different emission spectra or minimally overlapping emission spectra.

In some embodiments, the first and/or second extended capture probes 706,708 undergo second strand synthesis using the primer oligonucleotide probe 710 to generate the plurality of labeled oligonucleotide probes 712. In some embodiments, the plurality of labeled oligonucleotide probes 712 comprising the second label are substantially complementary to the first and/or second analytes 702,704, or a complement thereof. In some embodiments, a second strand synthesis helper probe (a partially double stranded DNA molecule capable of hybridizing to the 3′ end of the extended capture probe) can be ligated to the 3′ end of the first and/or second extended capture probes 706,708 (e.g., a first strand cDNA molecule) using a ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, Wis.), and SplintR (available from New England Biolabs, Ipswich, Mass.).

In some embodiments, the methods disclosed herein include detecting a first intensity of the first label and a second intensity of the second label of the plurality of labeled oligonucleotides hybridized to the extended capture probe, thereby detecting a length of the original analyte and determining the RNA integrity of the biological sample. In some embodiments, the primer oligonucleotide probe 710 serves as an indicator for the absolute number of analytes (e.g., transcripts). In some embodiments, a single primer oligonucleotide probe 710 anneals to a single extended capture probe. For example, in some embodiments, the fluorescence intensity of the first label of the primer oligonucleotide probe 710 is proportional to the number of analytes captured on the surface of the slide. In some embodiments, the fluorescence intensity of the second label of the labeled oligonucleotide probes 712 is indicative of the length of the labeled oligonucleotide probes 712, where shorter strands have reduced fluorescence and longer strands have increased fluorescence. However, by imaging the second label alone, the user is oftentimes unable to differentiate between strand length and absolute number of analytes captured. Thus, in some embodiments, the methods disclosed herein include calculating a ratio of the intensity of the second fluorescent label (e.g., second intensity) to the intensity of the first fluorescent label (e.g., first intensity) in order to normalize the fluorescence signal per captured analyte. In some embodiments, the first intensity is indicative of a capture of the analyte. In some embodiments, the second intensity is indicative of the length of the analyte. In some embodiments, the second intensity is an approximation of the length of the analyte. In some embodiments, the methods include an alternative quantitative assessment of the plurality of the first extended capture probe 706 and/or the second extended capture probe 708. For example, in some embodiments, the first extended capture probe 706 and/or the second extended capture probe 708 can be removed and quantified by various systems, such as polymerase chain reaction (e.g., quantitative PCR).

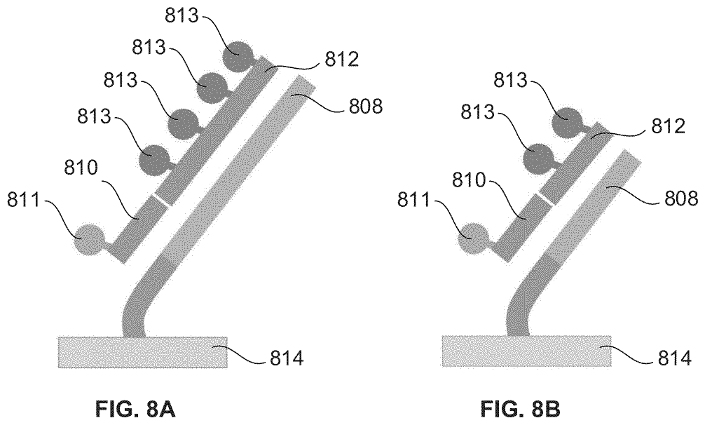

FIGS. 8A and 8B are schematics showing an example of how fluorescence intensity varies based on analyte or extended capture probe length. In these figures, a pair of second extended capture probes 808 having different lengths are shown. FIG. 8A shows the second extended capture probe 808 hybridized to a labeled oligonucleotide probe 812 and attached to the substrate 814. The labeled oligonucleotide probe 812 has a plurality of second labels 813 and a primer oligonucleotide probe 810 having a first label 811. In this example, the longer sequence of the extended capture probe incorporates four second labels 813 for every first label 811. Thus, a ratio of 4:1 is demonstrated. In some embodiments, the first label 811 serves as a marker for the extended captured probe 808. In some embodiments, absence of the first label 811 indicates that there is no captured analyte from the biological sample. In some embodiments, the second labels 813 serve as direct markers of the length of the labeled oligonucleotide probe. For example, in some embodiments, the longer the length of the labeled oligonucleotide probe, the higher incorporation of the second label 813, which in turn leads to an increase in the quantal yield of the fluorescence intensity signal.

FIG. 8B shows a substantially similar second extended capture probe 808 as the second extended capture probe 808 of FIG. 8A but has a length that is less than the length of the second extended capture probe 808 of FIG. 8A. In this example, the shorter sequence of the extended capture probe incorporates two second labels 813 for every first label 811. Thus, a ratio of 2:1 is demonstrated. In some embodiments, the fluorescence intensity of the second labels 813 of the longer, labeled oligonucleotide probe 812 depicted in FIG. 8A is greater than the fluorescence intensity of the second labels 813 of the shorter, labeled oligonucleotide probe 812 shown in FIG. 8B. In some examples, the calculated ratio of the detected fluorescence intensities of the first and second labels 811,813 of the longer, labeled oligonucleotide probe 812 depicted in FIG. 8A is greater than the calculated ratio of the detected fluorescence intensities of the first and second labels 811,813 of the shorter, labeled oligonucleotide probe 812 depicted in FIG. 8B. As such, a user can determine the length of an analyte based on the calculated ratio of the labeled oligonucleotide probe. In some embodiments, the methods disclosed herein further include calculating a ratio of a length of an 18S rRNA and/or a 28S rRNA. In some embodiments, the RNA integrity is high when the ratio of a second fluorescence intensity (e.g., second label 813) to a first fluorescence intensity (e.g., first label 811) is about 3:1 to about 10:1 (e.g., about 3:1 to about 4:1, about 4:1 to about 5:1, about 5:1 to about 6:1, about 6:1 to about 7:1, about 7:1 to about 8:1, about 8:1 to about 9:1, about 9:1 to about 10:1, or more). In some embodiments, the RNA integrity is low when the ratio of the second fluorescence intensity (e.g., second label 813) to the first fluorescence intensity (e.g., first label 811) is less than about 2:1 (e.g., about 2:1 to about 1:1 or less). In some embodiments, the RNA integrity is high when the ratio of the second fluorescence intensity (e.g., second label 813) to the first fluorescence intensity (e.g., first label 811) is at least about 2:1. In some embodiments, the RNA integrity is high when the ratio of 28S rRNA to 18S rRNA is at least about 2:1.

In some embodiments, the primer oligonucleotide probe has a length that ranges from about 50 nucleotides to about 100 nucleotides (e.g., about 50 nucleotides to about 55 nucleotides, about 55 nucleotides to about 60 nucleotides, about 60 nucleotides to about 65 nucleotides, about 65 nucleotides to about 70 nucleotides, about 70 nucleotides to about 75 nucleotides, about 75 nucleotides to about 80 nucleotides, about 80 nucleotides to about 85 nucleotides, about 85 nucleotides to about 90 nucleotides, about 90 nucleotides to about 95 nucleotides, about 95 nucleotides to about 100 nucleotides, or more). In some embodiments, each labeled oligonucleotide probe comprising the second label has a length that ranges from about 50 nucleotides to about 100 nucleotides (e.g., about 50 nucleotides to about 55 nucleotides, about 55 nucleotides to about 60 nucleotides, about 60 nucleotides to about 65 nucleotides, about 65 nucleotides to about 70 nucleotides, about 70 nucleotides to about 75 nucleotides, about 75 nucleotides to about 80 nucleotides, about 80 nucleotides to about 85 nucleotides, about 85 nucleotides to about 90 nucleotides, about 90 nucleotides to about 95 nucleotides, about 95 nucleotides to about 100 nucleotides, or more).

In some embodiments, each labeled oligonucleotide probe comprising the second label of the plurality of labeled oligonucleotide probes has a length that is about 5% to about 20% (e.g., about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, about 19% to about 20%, or more) of a length of the extended capture probe.

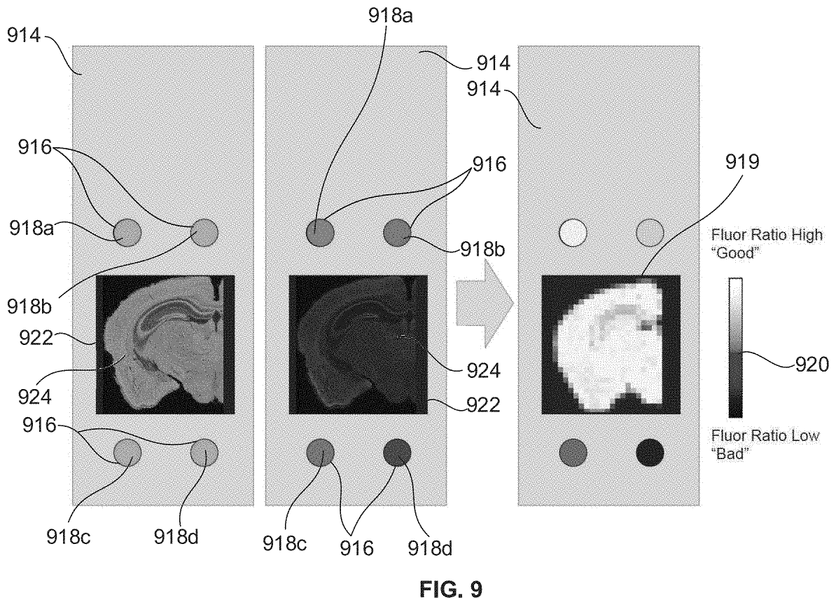

FIG. 9 shows an example of a visual representation of RNA integrity using ratiometric imaging of a biological sample. In some embodiments, the methods disclosed herein include placing the biological sample 924 on the substrate 914, within the boundaries of a sample region 922. In some embodiments, the substrate has four reference wells 916 configured to hold one or more reference analytes 918. In some embodiments, the reference analytes have a known length. In some embodiments, the reference analytes have different lengths (e.g., one or more varying lengths). FIG. 9 shows a first, second, third, and fourth reference analytes 918a-d having four different lengths that can, consequently, have four different fluorescence intensity signals.

In some embodiments, the methods disclosed herein include generating an image of the extended capture probe and using the image of the extended capture probe to generate a spatial nucleic acid (e.g., RNA) integrity number for a location on the substrate. In some embodiments, the imaging can be achieved by using a simple imaging device setup (including e.g., a microscope, such as an epifluorescent microscope, a charge-coupled device (CCD) camera, one or more optical filter sets, and/or a computer). In some embodiments, the imaging device detects, records, and/or transmits one or more fluorescence intensity signals to a computer.

In some embodiments, the spatial RNA integrity number can be determined by calculating the ratio of the first detected fluorescence intensity signal to the second detected fluorescence intensity signal (e.g., the fluorescence intensity signal of the first label of the primer oligonucleotide probe to the fluorescence intensity signal of the second label of the oligonucleotide probe) in a determined area of the image (e.g., a pixel). In some embodiments, the determined area of the image has an area ranging from about 0.1 millimeters (mm) to about 10 mm (e.g., about 0.1 mm to about 0.2 mm, about 0.2 mm to about 0.3 mm, about 0.3 mm to about 0.4 mm, about 0.4 mm to about 0.5 mm, about 0.5 mm to about 1 mm, about 1 mm to about 2 mm, about 2 mm to about 3 mm, about 3 mm to about 4 mm, about 4 mm to about 5 mm, about 5 mm to about 10 mm). For example, as shown in FIG. 9, the left and right substrates on the left side of the arrow show images of the biological samples 924 and reference analytes 918 as imaged using two different excitation and/or emission filters. In some embodiments, the methods disclosed herein include imaging the extended capture probes and reference analytes with one or more excitation and/or emission filters.

In some embodiments, determination of the fluorescence intensity ratio is performed computationally. In some embodiments, the calculated value of the ratio for each determined area (e.g., of each pixel) can then be compared to a fluorescence intensity value of one or more reference analytes 918, which have a known length and corresponding ratio of the first fluorescence intensity signal to the second fluorescence intensity signal. In some embodiments, the methods of the disclosure include using the image of the extended capture probe to generate a heat map 919 and reference scale 920. In some embodiments, the heat map 919 can be generated by assigning a specific color hue and/or color intensity to the values of the fluorescence intensity ratios, as shown in the reference scale 920, and superimposing that data on a reference image (e.g., an image of the biological sample). In some embodiments, the heat map 919 correlates the detected fluorescence intensity signals of the first and/or second labels of the extended capture probes with a length of the analyte (e.g., 18S rRNA and/or 28S rRNA). In some embodiments, a higher fluorescence intensity is correlated with a greater length of the analyte thereby indicating a better RNA integrity. For example, the reference scale 920 correlates darker color intensities to shorter analyte lengths and lighter color intensities to longer analyte lengths. Thus, the example heat map 919 shows most areas of the biological sample as having optimal (e.g., high) RNA integrity (lighter color intensities) and a few areas of the biological sample as having sub-optimal (e.g., low) RNA integrity (darker color intensities). In some embodiments, the methods disclosed herein provide an approximate RNA quality assessment of the biological sample while retaining the spatial positioning and anatomy of the biological sample (e.g., tissue section).

In some embodiments, the methods disclosed herein include determining RNA integrity by calculating a ratio of the length of a first analyte (e.g., 18S rRNA) to the length of a second analyte (e.g., 28S rRNA). In some embodiments, the RNA integrity is high when the ratio of the length of a first analyte (e.g., 18S rRNA) to a second analyte (e.g., 28S rRNA) is greater than or equal to about 2.

In some embodiments, an increase in the length of the analyte is correlated to an increase of the RNA integrity of the biological sample. In some embodiments, the RNA integrity is high when the length of the analyte is about 50% to about 90% (e.g., 50% to about 55%, 55% to about 60%, 60% to about 65%, 65% to about 70%, 70% to about 75%, 75% to about 80%, 80% to about 85%, 85% to about 90%, or more) of an intact length of the analyte. In some embodiments, the RNA integrity is high when the length of the analyte is about 900 to about 1600 nucleotides (e.g., about 900 nucleotides to about 950 nucleotides, about 950 nucleotides to about 1000 nucleotides, about 1000 nucleotides to about 1050 nucleotides, about 1050 nucleotides to about 1100 nucleotides, about 1100 nucleotides to about 1150 nucleotides, about 1150 nucleotides to about 1200 nucleotides, about 1200 nucleotides to about 1250 nucleotides, about 1250 nucleotides to about 1300 nucleotides, about 1300 nucleotides to about 1350 nucleotides, about 1350 nucleotides to about 1400 nucleotides, about 1400 nucleotides to about 1450 nucleotides, about 1450 nucleotides to about 1500 nucleotides, about 1500 nucleotides to about 1550 nucleotides, about 1550 nucleotides to about 1660 nucleotides, or more).

In some embodiments, the RNA integrity is high when the length of the analyte is about 2500 to about 4500 nucleotides (e.g., about 2500 nucleotides to about 2600 nucleotides, about 2600 nucleotides to about 2700 nucleotides, about 2700 nucleotides to about 2800 nucleotides, about 2800 nucleotides to about 2900 nucleotides, about 2900 nucleotides to about 3000 nucleotides, about 3000 nucleotides to about 3100 nucleotides, about 3100 nucleotides to about 3200 nucleotides, about 3200 nucleotides to about 3300 nucleotides, about 3300 nucleotides to about 3400 nucleotides, about 3400 nucleotides to about 3500 nucleotides, about 3500 nucleotides to about 3600 nucleotides, about 3600 nucleotides to about 3700 nucleotides, about 3700 nucleotides to about 3800 nucleotides, about 3800 nucleotides to about 3900 nucleotides, about 3900 nucleotides to about 4000 nucleotides, about 4000 nucleotides to about 4100 nucleotides, about 4100 nucleotides to about 4200 nucleotides, about 4200 nucleotides to about 4300 nucleotides, about 4300 nucleotides to about 4400 nucleotides, about 4400 nucleotides to about 4500 nucleotides, or more).

(c) FRET Methods

A method directed to capturing an analyte of a biological sample on the surface of a substrate and determining a length of the analyte may be substantially similar in several aspects to the relative fluorescent signal methods discussed above but can include an alternative detection step (e.g., an alternative way of determining RNA integrity). In some embodiments, the methods include a first labeled oligonucleotide probe that is capable of transferring energy to a second labeled oligonucleotide probe by a dipole-dipole coupling mechanism. In some embodiments, the dipole-dipole coupling mechanism is a Forster resonance energy transfer (FRET). For example, the first and second labeled oligonucleotide probes can comprise detectable labels that can be used as a FRET system. Such FRET-labeled oligonucleotide probes can allow a user to determine the energy transferred from the first labeled oligonucleotide probe to the second labeled oligonucleotide probe, thereby detecting a length of the analyte and determining the RNA integrity of the biological sample.

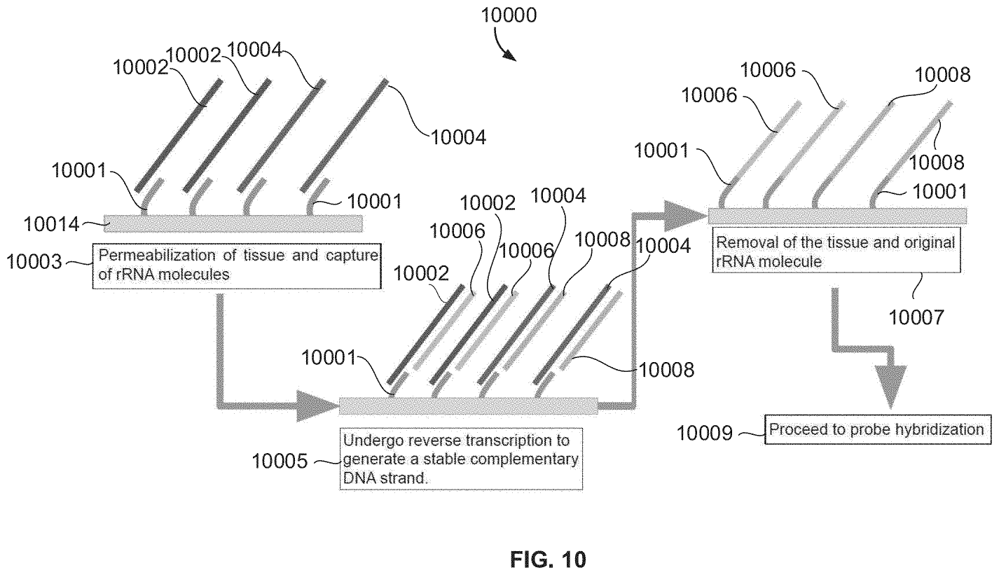

FIGS. 10-12B illustrate an example method to determine the length of the analyte via the detection of energy transferred from the first labeled oligonucleotide probe to the second labeled oligonucleotide probe. In some embodiments, an increased FRET efficiency (relative to a reference) of the first and second labeled oligonucleotide probes in a biological sample can indicate that the biological sample has a higher RNA integrity. Accordingly, in some embodiments, a decreased FRET efficiency (relative to a reference) of the first and second labeled oligonucleotide probes in a biological sample can indicate that the biological sample has a lower RNA integrity. As used herein, the term “FRET efficiency” refers to the proportion of the first labeled oligonucleotide probes (e.g., donor molecules) that have transferred an excitation state energy to the second labeled oligonucleotide probes (e.g., acceptor molecules).

FRET occurs between two labeled probes (e.g., comprising fluorophores) in close proximity with substantial overlap (e.g., greater than 30%) between the donor's emission and acceptor's absorption spectra and is characterized by the FRET efficiency (E). FRET E refers to the percent of energy transfer from the donor to acceptor fluorophores at a given state and can be quantitatively described by the following equations:

E=1/(1+r⁶/r₀⁶)

r₀=0.02108(κ²ϕ_{D}n⁻⁴(∫₀^{∞}f_{D}(λ)ε_{A}(λ)λ⁴dλ))^{1/6}(in nm)

where r is the distance between donor and acceptor dipoles; r₀ is the distance at which the FRET E is 50%; and κ² is the inter-dipole orientation factor (assumed to be ⅔ corresponding to a random orientation), n is the refractive index of the medium surrounding the fluorophores; φ_{D} is the quantum yield (QY) of the donor in absence of the acceptor; f_{D}(λ) is the (wavelength dependent) corrected donor fluorescence intensity at wavelength λ with the total intensity (area under the curve) normalized to unity and is dimensionless; EA is the (wavelength dependent) extinction coefficient (EC) of the acceptor (in M⁻¹·cm⁻¹), and λ is the wavelength, whereby the integral term represents the spectral overlap between the donor emission and the acceptor excitation. For a given FRET pair, the FRET E is proportional to the inverse sixth power of the distance between two fluorophores.

In some embodiments, the extended capture probes include “FRET labels,” which typically comprise at least two probes (e.g., chromophores or fluorophores) that engage in FRET such that at least a portion of the energy absorbed by at least one donor fluorophore is transferred to at least one acceptor fluorophore which emits at least a portion of the transferred energy as a detectable signal contributing to an emission spectrum. In certain embodiments, at least two probes in a FRET label emit detectable signals that contribute to a resulting emission spectrum comprising at least two peaks. Such a FRET label can be termed a “multi-spectral” construct (or a “dual-spectral” construct when the emission spectrum has only two peaks). In some embodiments, the probes are configured to achieve a desired FRET efficiency. In some embodiments, the desired FRET efficiency is designed to ensure a desired emission intensity (or range thereof) at one or more emission wavelengths. In some embodiments, one or more FRET labels is present in a single extended capture probe. In some embodiments, a first FRET label has an emission spectrum that is distinguishable from the emission spectrum of a second FRET label in the extended capture probe such that the identity of each FRET label can be unambiguously determined. In some embodiments, the emission spectra of certain FRET labels are distinguishable from one another due to variations in emission intensity at one or more wavelengths as a result of variations in FRET efficiency. In some embodiments, the FRET labels comprise the same set of probes (e.g., fluorophores or chromophores), but have a different configuration (e.g., hybridization arrangement) and therefore, different emission spectra based at least in part on different FRET efficiencies.

Referring to FIG. 10, a method (10000) of determining RNA integrity of a biological sample (e.g., a tissue sample) is substantially similar in several aspects and steps to the method (700) of determining RNA integrity of a biological sample via relative fluorescent signal methods, except that the first and second labeled oligonucleotide probes hybridized to the extended capture probe are capable of transferring energy via a dipole-dipole coupling mechanism (e.g., FRET).

In some embodiments, the method (10000) of determining RNA integrity of a biological sample (e.g., a tissue sample) initially involves contacting the biological sample with a substrate 10014 comprising a plurality of capture probes 10001. In some embodiments, the biological sample comprises a tissue section, a region within a tissue, or a single cell within a tissue. In some embodiments, the capture probes 10001 are designed to hybridize to one or more desired analytes of the biological sample. For example, as shown in FIG. 10, the capture probes 10001 can be designed to hybridize to a first analyte 10002 and a second analyte 10004. In some embodiments, the first analyte 10002 and/or the second analyte 10004 are substantially similar in composition and function to the first analyte 702 and the second analyte 704 discussed above.

In some embodiments, the biological sample is permeabilized while on the substrate 10014 (e.g., by providing a solution comprising a permeabilization reagent to the biological sample), and analytes from the biological sample are contacted with one or more capture probes 10001 (step 10003). For example, permeabilization of a biological sample can facilitate analyte release and capture. Example permeabilization agents and conditions are described in Section (I)(d)(ii)(13) or the Exemplary Embodiments Section of WO 2020/176788 and/or U.S. Patent Application Publication No. 2020/0277663. In some embodiments, one or more capture probes 10001 (e.g., in an array) capture the first analyte 10002 and/or the second analyte 10004. In some embodiments, one or more capture probes 10001 include a capture domain (e.g., a poly(T) sequence, a poly(A) sequence, a non-homopolymeric sequence). In some embodiments, the capture domain includes a sequence complementary to the first analyte 10002 and/or the second analyte 10004. For example, one or more capture domains can hybridize to poly(A) tails of mRNA molecules. In some instances, one or more capture domains on the array can hybridize to a portion of rRNA molecules. In some embodiments, the capture domain comprises a poly(T) sequence. In some embodiments, one or more capture probes 10001 include a spatial barcode.

Reverse transcription can be carried out using a reverse transcriptase to generate a first extended capture probe 10006 and a second extended capture probe 10008 based on the captured, first and second analytes 10002, 10004, respectively (step 10005). For example, in some embodiments, the methods include extending a 3′ end of the capture probe 10001 using the first and/or second analytes 10002, 10004 as a template to generate the first and second extended capture probes 10006, 10008. In some embodiments, first and second extended capture probes 10006, 10008 are complementary DNA (cDNA) strands that are generated based on the captured, first and second analytes 10002, 10004, respectively. In some embodiments, reverse transcription allows for the generation of cDNA strands that stabilize the sequences on the substrate 10014, thereby allowing for subsequent processing. In some embodiments, a polynucleotide tail, e.g., a poly(A) tail, is incorporated at the 3′ end of the extended probe molecules. In some embodiments, the polynucleotide tail is incorporated using a terminal transferase active enzyme. In some embodiments, the sequence and location of the captured first and second analytes 10002,10004 can be determined (e.g., by sequencing the capture probe that contains the spatial barcode as well as the complementary cDNA).

The biological sample, including the first and second analytes 10002, 10004, is removed from the substrate 10014 leaving behind the first and second extended capture probes 10006,10008 (step 10007). In some embodiments, the removal step includes enzymatic and/or chemical degradation of cells of the biological sample. For example, the removal step can include treating the biological sample with an enzyme (e.g., a proteinase such as proteinase K) to remove at least a portion of the biological sample from the substrate 10014. In some embodiments, the removal step includes enzymatic removal of the original analyte (e.g., rRNA, mRNA, etc.) of the biological sample. For example, the removal step can include treating the biological sample with an enzyme (e.g., an RNase). In some embodiments, the removal step can include ablation of the tissue (e.g., laser ablation).

In some embodiments, the methods include an alternative quantitative assessment of the plurality of the first extended capture probe 10006 and/or the second extended capture probe 10008. For example, in some embodiments, the first extended capture probe 10006 and/or the second extended capture probe 10008 can be removed and quantified by various systems, such as polymerase chain reaction (e.g., quantitative PCR).

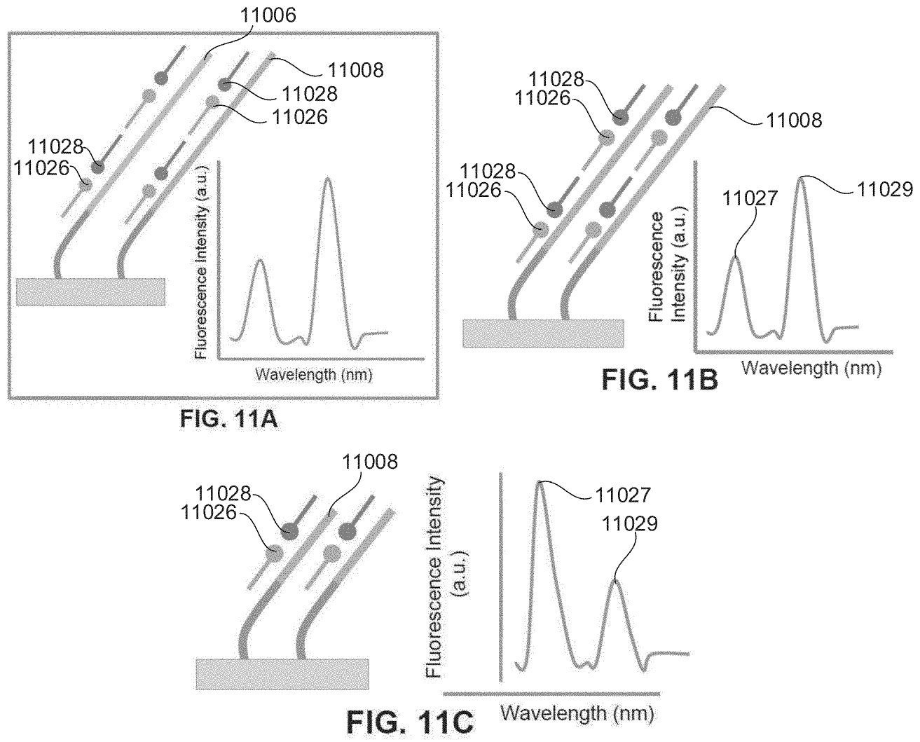

As shown in FIG. 11A, the method (10000) includes a first labeled oligonucleotide probe 11026 hybridizing a plurality of labeled oligonucleotide probes to the first extended capture probe 11006 and/or the second extended capture probe 11008. In some embodiments, the plurality of labeled oligonucleotide probes comprises FRET probe. For example, in some embodiments, the first extended capture probe 11006 includes an acceptor molecule and the second extended capture probe 11008 includes a donor molecule. In some embodiments, the first extended capture probe 11006 includes a donor molecule and the second extended capture probe 11008 includes an acceptor molecule. In some embodiments, the acceptor molecule is an organic dye including but not limited to, cy3, rhodamine, cy5, a biological fluorophore including but not limited to enhanced yellow fluorescent protein (EYFP), mVenus sEYFP, mCitrine, YPet, mRuby2, mRuby3, mCherry, IFP1.4m, iRFPm, a quantum dot (QD) including but not limited to cadmium selenide zinc sulfide (CdSe/ZnS) QD, cadmium selenide zinc tellurium (CdSe/ZnTe) QD, indium phosphide zinc sulfide (InP/ZnS) QD, lead sulfide (PbS) core-type QD, perovskite, or any combination thereof. In some embodiments, the donor molecule is an organic dye including but not limited to fluorescein, coumarin, a biological fluorophore including but not limited to aquamarine, enhanced cyan fluorescent protein (ECFP), mTurquoise2, mCerulean3, lumazine binding protein (LUMPm), monomeric teal fluorescent protein 1 (mTFP1), enhanced green fluorescent protein (EGFP), NowGFP, Clover, Clover3, mNeonGreen, mPlum, eqFP650, mCardinal, a quantum dot including but not limited to CdSe/ZnS QD, CdSe/ZnTe QD, InP/ZnS QD, PbS core-type QD, perovskite, or any combination thereof.

In some embodiments, the distance between the first labeled oligonucleotide probe 11026 and the second labeled oligonucleotide probe 11028 ranges from about 1 nanometer (nm) to about 10 nm (e.g., about 1 nm to about 2 nm, about 2 nm to about 3 nm, about 3 nm to about 4 nm, about 4 nm to about 5 nm, about 5 nm to about 6 nm, about 6 nm to about 7 nm, about 7 nm to about 8 nm, about 8 nm to about 9 nm, about 9 nm to about 10 nm). In some embodiments, the first labeled oligonucleotide probe 11026 and the second labeled oligonucleotide probe 11028 are hybridized in an alternating pattern to the extended capture probe 11008. In some embodiments, the alternating pattern maximizes the FRET efficiency.

FIGS. 11B and 11C are schematics showing an example of how FRET efficiency varies based on the length of an analyte and/or extended capture. In these figures, the second extended capture probes 11008 of FIG. 11B have a greater length than the length of the second extended capture probes 11008 of FIG. 11C. The extended capture probes 11008 of FIG. 11B have an increased number of acceptors and donors that result in an increased FRET efficiency. In some embodiments, the increased FRET efficiency has an impact on the spectral curves generated by the biological sample, where the emission intensity of the donor 11027 is reduced, and the emission intensity of the acceptor 11029 is increased, as shown in the graph of FIG. 11B. In contrast, when the extended capture probes have a decreased length, the extended capture probes 11028 have a decreased number of acceptors and donors that results in a reduced FRET efficiency. In some embodiments, the decreased FRET efficiency has an impact on the spectral curves generated by the biological sample, where the emission intensity of the donor 11027 is increased, and the emission intensity of the acceptor 11029 is decreased, as shown in the graph of FIG. 11C.

In some embodiments, the FRET efficiency of the longer, extended capture probe 11008 depicted in FIG. 11B is greater than the FRET efficiency of the shorter, extended capture probe 11008 shown in FIG. 11C. In some embodiments, analytes having shorter lengths or fragmented analytes generate lower FRET efficiencies compared to the FRET efficiencies generated by analytes having longer lengths or more intact sequences. In some embodiments, the methods disclosed herein include measuring the FRET efficiency of the first and second labeled oligonucleotide probes 11026, 11028. In some embodiments, FRET efficiency determination can be performed by using a fluorescence spectrometer detector and/or camera and computationally. Accordingly, the methods disclosed herein include determining the length of an analyte based on the measured FRET efficiency of the extended capture probes. In some embodiments, the RNA integrity is high when the FRET efficiency ranges from about 70% to about 100%.

In some embodiments, the first labeled oligonucleotide probe and the second labeled oligonucleotide probe have a length that ranges from about 50 nucleotides to about 100 nucleotides (e.g., about 50 nucleotides to about 55 nucleotides, about 55 nucleotides to about 60 nucleotides, about 60 nucleotides to about 65 nucleotides, about 65 nucleotides to about 70 nucleotides, about 70 nucleotides to about 75 nucleotides, about 75 nucleotides to about 80 nucleotides, about 80 nucleotides to about 85 nucleotides, about 85 nucleotides to about 90 nucleotides, about 90 nucleotides to about 95 nucleotides, about 95 nucleotides to about 100 nucleotides, or more).

In some embodiments, the first labeled oligonucleotide probe 11026 and the second labeled oligonucleotide probe 11028 have a length that is about 1% to about 10% of a length of the extended capture probe (e.g., about 1% to about 2%, about 2% to about 3%, about 3% to about 4%, about 4% to about 5%, about 5% to about 6%, about 5% to about 6%, about 6% to about 7%, about 7% to about 8%, about 8% to about 9%, about 9% to about 10%, or more).

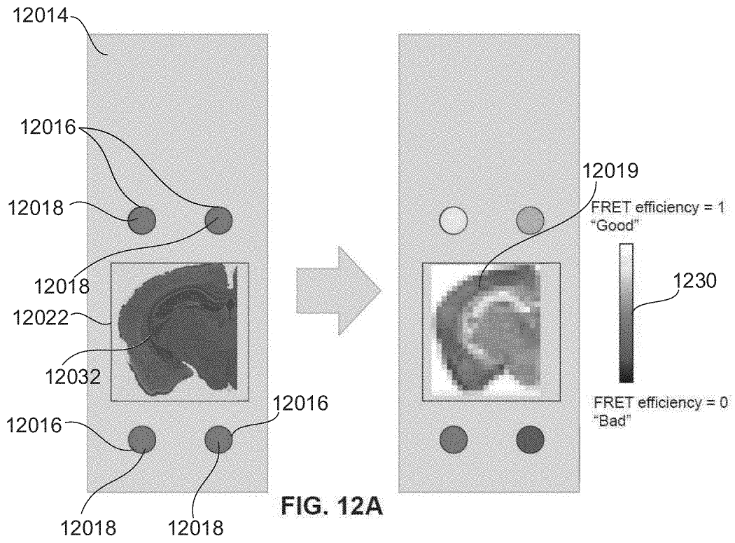

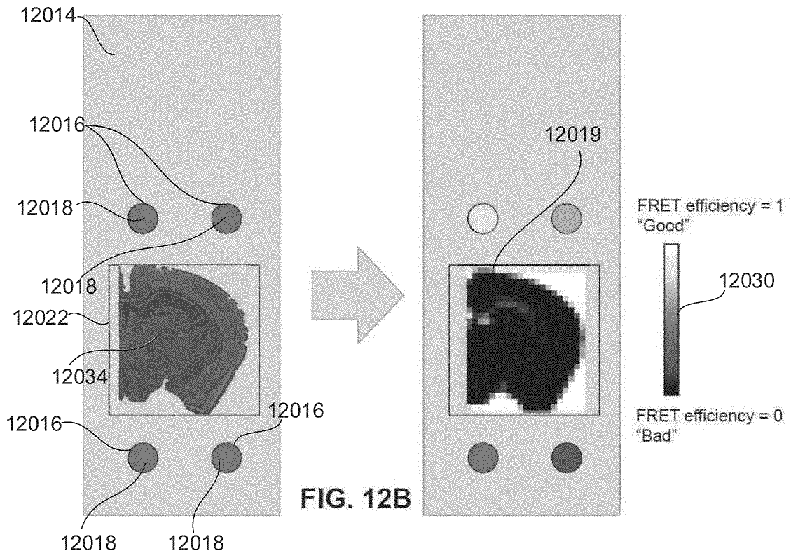

Referring to FIGS. 12A and 12B, example visual representations of RNA integrity of a biological sample are substantially similar in several aspects to the visual representation of RNA integrity using ratiometric imaging shown in FIG. 9, except that the visual representations of FIGS. 12A and 12B are example visual representations obtained by calculating the FRET efficiency between first and second labeled oligonucleotide probes. Similarly to FIG. 9, FIG. 12A shows an example heat map 12019 where most areas of the biological sample are shown to have an optimal (e.g., high) RNA integrity (lighter color intensities), and a few areas of the biological sample are shown to have sub-optimal (e.g., low) RNA integrity (darker color intensities). In contrast, FIG. 12B shows an example heat map 12019 where most areas of the biological sample are shown to have a sub-optimal (e.g., low) RNA integrity (darker color intensities), and a few areas of the biological sample are shown to have optimal (e.g., high) RNA integrity (lighter color intensities).

(d) Methods of Performing RNA Analysis

In some embodiments, after determining the RNA integrity of a biological sample, RNA analysis can be performed on a related biological sample (e.g., a serial tissue section). In some embodiments, RNA analysis can be performed on the related biological sample if the RNA integrity is determined to be a high RNA integrity. For example, when performing or prior to performing a workflow (e.g., RNA analysis workflow, spatial analysis workflow, an NGS workflow, or the like), a user can determine the RNA integrity of a biological sample using the methods described herein prior to continuing with preparation of a related sample for analysis (e.g., RNA analysis, NGS analysis, spatial analysis, etc.). In other words, a user can use the RNA integrity results as a quality assessment tool to assess the molecules of interest (e.g., RNA or other biological molecules) in the sample are sufficiently viable (e.g., not fragmented) prior to subjecting related biological samples to further analysis. In some embodiments, the RNA integrity results provided by the methods and devices of the disclosure can help a user determine the quality of a sample and whether or not to proceed with downstream sample analysis based on the determined quality (e.g., RNA integrity) of the biological sample. In some embodiments, RNA analysis may not be performed on the related biological sample if the RNA integrity is determined to be a low RNA integrity.

In some instances, the related biological sample is placed on a second substrate and analytes are captured using methods disclosed herein. The methods include determining abundance and location of a plurality of analytes in a related biological sample. In some examples, the determining the abundance and location includes: (a) contacting a second spatial array comprising a comprising a plurality of second spatial capture probes with the related (e.g., serial) tissue section, wherein a second spatial capture probe of the plurality of second spatial capture probes comprises a second spatial barcode and a second spatial capture domain, (b) hybridizing an analyte from the related (e.g., serial) tissue section to a second spatial capture domain, and (c) determining: (i) all or a part of a sequence of the analyte from the related (e.g., serial) tissue section, or a complement thereof, and (ii) the spatial barcode, or a complement thereof, and using the determined sequence of (i) and (ii) to determine the abundance and the location of the analyte from the serial tissue section.

The methods of RNA analysis include preparation of the related biological sample, which includes but is not limited to sectioning the sample, staining and deparaffinizing the sample, imaging the sample, and permeabilizing the sample. In some instances, permeabilization includes treatment with a solution comprising proteinase K, pepsin, or a combination of both. Preparation of biological samples is disclosed in WO 2020/176788 and/or U.S. Patent Application Publication No. 2020/0277663, each of which is incorporated by reference in its entirety.

In some instances, analytes are captured by capture probes on the second spatial array. After an analyte from the sample has hybridized or otherwise been associated with a capture probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the sample with an array comprising a plurality of capture probes, wherein a capture probe of the plurality captures the analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by uracil-DNA glycosylase, apurinic/apyrimidinic (AP) endonuclease (APE1), uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, Wis.). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N^{M} DNA ligase, New England Biolabs), Ampligase™ (available from Lucigen, Middleton, Wis.), and SplintR (available from New England Biolabs, Ipswich, Mass.). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, or about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, or about 99 probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the analyte and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze barcoded analyte. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### III. Compositions for Determining RNA Integrity

(a) Substrates

(i) Background

For the spatial array-based analytical methods described herein, a substrate functions as a support for direct or indirect attachment of capture probes to features of the array. In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and which allows for positioning of biological samples, analytes, features, and/or capture probes on the substrate.

Further, a “substrate” as used herein, and when not preceded by the modifier “chemical,” refers to a member with at least one surface that generally functions to provide physical support for biological samples, analytes, and/or any of the other chemical and/or physical moieties, agents, and structures described herein. Substrates can be formed from a variety of solid materials, gel-based materials, colloidal materials, semi-solid materials (e.g., materials that are at least partially cross-linked), materials that are fully or partially cured, and materials that undergo a phase change or transition to provide physical support. Examples of substrates that can be used in the methods and systems described herein include, but are not limited to, slides (e.g., slides formed from various glasses, slides formed from various polymers), hydrogels, layers and/or films, membranes (e.g., porous membranes), flow cells, cuvettes, wafers, plates, or combinations thereof. In some embodiments, substrates can optionally include functional elements such as recesses, protruding structures, microfluidic elements (e.g., channels, reservoirs, electrodes, valves, seals), and various markings, as will be discussed in further detail below.

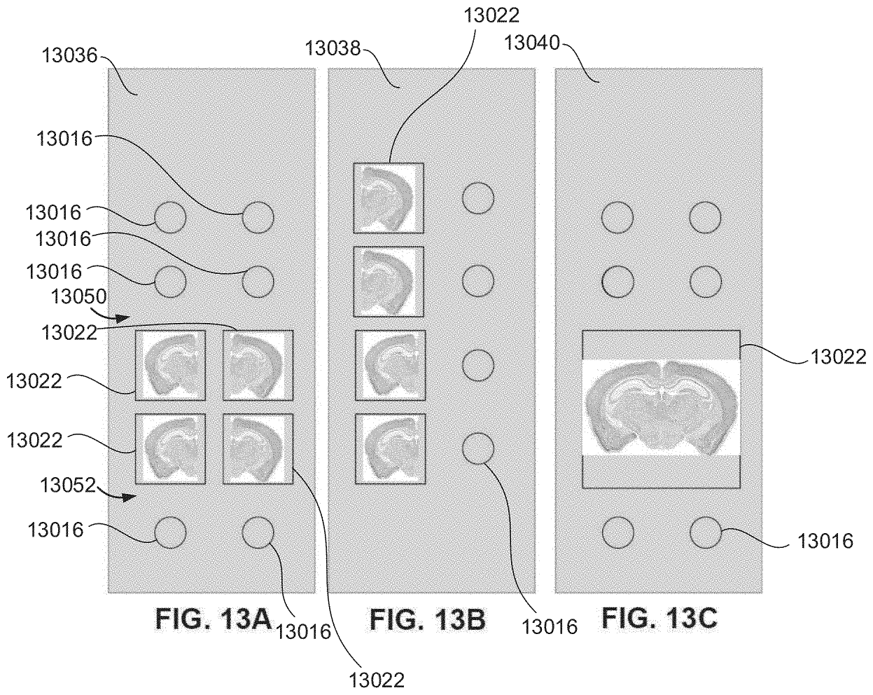

FIGS. 13A-13C show example substrates 13036, 13038, and 13040, which include different number and configurations of the sample regions 13022 and reference wells 13016. Referring to FIG. 13A, the substrate 13036 includes four sample regions 13022 having a squared shape and configured to receive a biological sample. The sample regions 13022 are arranged in a grid-like pattern (e.g., forming a square) without overlapping and abutting each other. In some embodiments, a capture probe is attached to a location on the sample region. The substrate 13036 further includes six reference wells 13016 or reservoirs defined by a surface of the substrate and having a circular shape and positioned near a first edge 13050 (e.g., a top edge) and a second edge 13052 (e.g., a bottom edge) of the sample region grid.

The reference wells 13016 are configured to receive a reference analyte (e.g., a fluorescently labeled oligonucleotide) having a known length and/or a corresponding fluorescence intensity value or FRET efficiency value. Non-limiting examples of reference analytes include 18S rRNA, 28S rRNA, tRNA, miRNA, snRNA, and/or ribozymes. In some embodiments, the known length of the reference analyte ranges from about 10 nucleotides to about 1800 nucleotides (e.g., about 10 nucleotides to about 100 nucleotides, about 100 nucleotides to about 200 nucleotides, about 200 nucleotides to about 300 nucleotides, about 300 nucleotides to about 400 nucleotides, about 400 nucleotides to about 500 nucleotides, about 500 nucleotides to about 600 nucleotides, about 600 nucleotides to about 700 nucleotides, about 700 nucleotides to about 800 nucleotides, about 800 nucleotides to about 900 nucleotides, about 900 nucleotides to about 1000 nucleotides, about 1000 nucleotides to about 1100 nucleotides, about 1100 nucleotides to about 1200 nucleotides, about 1200 nucleotides to about 1300 nucleotides, about 1300 nucleotides to about 1400 nucleotides, about 1400 nucleotides to about 1500 nucleotides, or more). In some embodiments, the known length of the reference analyte ranges from about 10 nucleotides to about 5000 nucleotides (e.g., about 10 nucleotides to about 1000 nucleotides, about 1000 nucleotides to about 2000 nucleotides, about 2000 nucleotides to about 3000 nucleotides, about 3000 nucleotides to about 4000 nucleotides, about 4000 nucleotides to about 5000 nucleotides, or more).

Referring to FIG. 13B, the substrate 13038 includes four sample regions 13022 having a squared shape and configured to receive a biological sample. The sample regions 13022 are arranged in a linear pattern without overlapping and abutting each other. The substrate 13038 further includes four reference wells 13016 having a circular shape, and each reference well is positioned directly across from a sample region 13022.

Referring to FIG. 13C, the substrate 13040 includes one sample region 13022 having a squared shape and configured to receive a biological sample. The sample region 13022 is positioned off-center on the substrate 13040; however, in some embodiments, the sample region 13022 can be positioned on any suitable portion of the substrate 13040. The substrate 13040 further includes six reference wells 13016 having a circular shape and positioned around a first and second edge of the sample region 13022.

In some embodiments, the substrates disclosed herein have about 1 sample region to about 12 sample regions (e.g., about 1 sample region to about 2 sample regions, about 2 sample regions to about 3 sample regions, about 3 sample regions to about 4 sample regions, about 4 sample regions to about 5 sample regions, about 5 sample regions to about 6 sample regions, about 6 sample regions to about 7 sample regions, about 7 sample regions to about 8 sample regions, about 8 sample regions to about 9 sample regions, about 9 sample regions to about 10 sample regions, about 10 sample regions to about 11 sample regions, about 11 sample regions to about 12 sample regions) or more. In some embodiments, the substrates disclosed herein have any suitable number of sample regions configured to receive a biological sample. In some embodiments, the substrates disclosed herein do not require a sample region and instead, a biological sample may be placed on any suitable portion of the surface of the substrate. In some embodiments, the sample region(s) are substantially squared, rectangular, circular, triangular, or possess any other suitable shape or are amorphous. In some embodiments, the sample region has an area ranging from about at least 0.4 centimeters squared (cm²) to about 20.0 cm² (e.g., about 0.4 cm² to about 0.6 cm², about 0.6 cm² to about 0.8 cm², about 0.8 cm² to about 1.0 cm², about 1.0 cm² to about 2.0 cm², about 2.0 cm² to about 3.0 cm², about 3.0 cm² to about 4.0 cm², about 4.0 cm² to about 5.0 cm², about 5.0 cm² to about 5.0 cm², about 10.0 cm² to about 10.0 cm², about 10.0 cm² to about 15.0 cm², about 15.0 cm² to about 20.0 cm²).

In some embodiments, the substrates disclosed herein have about 1 reference well to about 12 reference wells (e.g., about 1 reference well to about 2 reference wells, about 2 reference wells to about 3 reference wells, about 3 reference wells to about 4 reference wells, about 4 reference wells to about 5 reference wells, about 5 reference wells to about 6 reference wells, about 6 reference wells to about 7 reference wells, about 7 reference wells to about 8 reference wells, about 8 reference wells to about 9 reference wells, about 9 reference wells to about 10 reference wells, about 10 reference wells to about 11 reference wells, about 11 reference wells to about 12 reference wells, or more). In some embodiments, the reference well defines a volume configured to receive an analyte having a known length. In some embodiments, the volume ranges from about at least 20 μL to about 150 μL (e.g., about 20 μL to about 30 μL, about 30 μL to about 40 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 110 μL, about 110 μL to about 120 μL, about 120 μL to about 130 μL, about 140 μL to about 150 μL).

In some embodiments, the substrates disclosed herein have any suitable number of reference wells configured to receive a biological sample. In some embodiments, the substrates disclosed herein do not include a reference well. For example, in some embodiments, one or more reference wells may be provided on a separate substrate. In some embodiments, the reference well(s) are substantially squared, rectangular, circular, triangular, or possess any other suitable shape or are amorphous. In some embodiments, the reference well has an area ranging from about 0.01 centimeters squared (cm²) to about 0.4 cm² (e.g., about 0.01 cm² to about 0.05 cm², about 0.05 cm² to about 0.10 cm², about 0.10 cm² to about 0.15 cm², about 0.15 cm² to about 0.20 cm², about 0.20 cm² to about 0.25 cm², about 0.25 cm² to about 0.30 cm², about 0.30 cm² to about 0.35 cm², about 0.35 cm² to about 0.40 cm²). In some embodiments, the reference wells 13016 and/or the sample regions 13022 are formed by modifying the surface of the substrates via stamping, microetching, or molding techniques. In some embodiments, the reference wells 13016 wells can be formed by one or more shallow depressions on the surface of the substrate.

In some embodiments, the reference wells or reservoirs are discrete reservoirs. In some embodiments, the reference wells or reservoirs are not interconnected with each other. In some embodiments, the reference wells or reservoirs are not interconnected with one or more sample regions. In some embodiments, the reference well or reservoir is a recess defined by the surface of the substrate. In some embodiments, the reference well or reservoir has an opening defined by the surface of the substrate and a bottom surface defined within the reference well or reservoir, the bottom surface directly opposing the opening. In some embodiments, the reference well or reservoir has one or more walls extending from the opening to the bottom surface, the one or more walls defining a depth of the reference well or reservoir. In some embodiments, the bottom surface is a concave surface, a convex surface, or a flat surface. In some embodiments, the depth of the reference well or reservoir is about equal to a radius of the opening. In some embodiments, the depth of the reference well or reservoir is greater than a radius of the opening. In some embodiments, the depth of the reference well or reservoir is less than a radius of the opening.

In some embodiments, the one or more walls may at least partially separate the reference well or reservoir from at least one other adjacent reference well or reservoir. In some embodiments, the one or more walls are perpendicular to a plane defined by the opening of the reference well or reservoir. In some embodiments, the one or more walls can extend vertically from a plane defined by the opening of the well or reservoir to the bottom surface. In some embodiments, the reference well or reservoir may not have a well-defined wall that is perpendicular to a plane defined by the opening (e.g., the bottom surface may extend in some manner directly to the opening without forming a wall perpendicular to the opening). In some examples, the bottom surface and the opening can be separated, with or without a wall, by a distance that is greater than, less than, or about equivalent to a radius of the opening.

A substrate may be substantially similar in construction and function in several aspects to the substrates 13036, 13038, 13040 discussed above, but can include one or more alternative reference arrays instead of the reference wells 13016. In some embodiments, the reference array may have a plurality of “spots” or micro-wells containing one or more reference analytes. Such array configurations can allow a user to include a plurality of reference analytes on the substrate to achieve optimal visualization (e.g., a heat map with optimal resolution) of the calculated RNA integrity values.

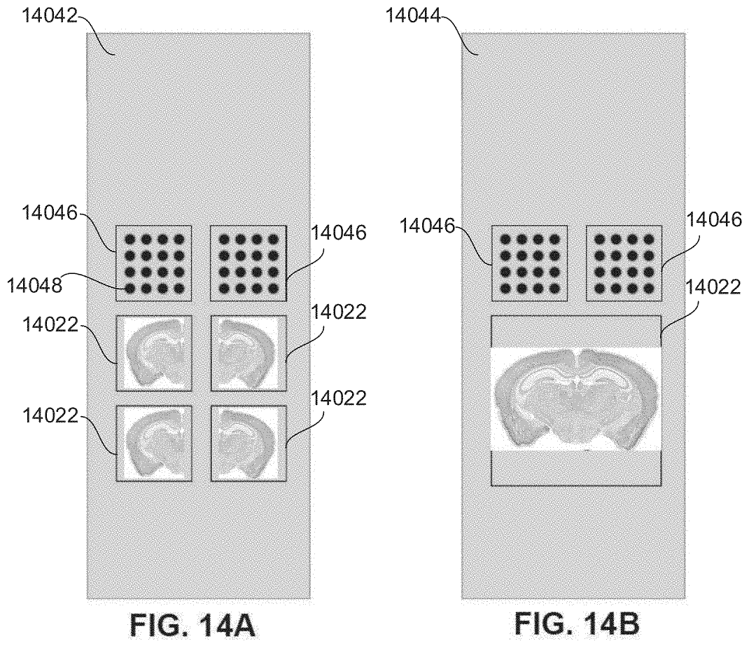

FIGS. 14A-14B illustrate examples of substrates 14042, 14044 that include reference two arrays 14046. The reference arrays 14046 are arranged linearly, in a horizontal row above (e.g., near a top edge of) one or more sample regions 14022. Each reference array 14046 includes sixteen reference analyte “spots” 14048 arranged in a grid-like pattern (e.g., a squared pattern). Each reference analyte spot 14084 includes a plurality of reference analytes of known length and corresponding fluorescence intensity values and/or FRET efficiency values. The reference analyte spots 14084 can provide a user a visual reference to compare an analyzed biological sample to. Additionally, the methods disclosed herein can use the plurality of reference analyte spots 14084 to generate a heat map that reflects the spatial RNA integrity values of the biological sample.

The reference arrays 14046 can be prepared by depositing features (e.g., droplets, beads) on a substrate surface to produce a spatially-barcoded array. Methods of depositing (e.g., droplet manipulation) features are known in the art (see, U.S. Patent Application Publication No. 2008/0132429, Rubina, A. Y., et al., Biotechniques. 2003 May; 34(5):1008-14, 1016-20, 1022 and Vasiliskov et al. Biotechniques. 1999 September; 27(3):592-4, 596-8, 600 passim. each herein incorporated by reference in its entirety). A reference analyte spot 14048 can be printed or deposited at a specific location on the substrate (e.g., inkjet printing). In some embodiments, each reference analyte spot 14048 can have a unique length that functions as a unique fluorescence intensity value. In some embodiments, a reference analyte spot 14048 can be printed or deposited at the specific location using an electric field. A reference analyte spot 14048 can contain a photo-crosslinkable polymer precursor and an oligonucleotide. In some embodiments, the photo-crosslinkable polymer precursor can be deposited into a patterned feature on the substrate (e.g., well).

In some embodiments, the reference array 14046 includes about 1 reference analyte spot 14048 to about 64 reference analyte spot 14048 or more (e.g., about 1 reference analyte spot to about 4 reference analyte spots, about 4 reference analyte spots to about 8 reference analyte spots, about 8 reference analyte spots to about 12 reference analyte spots, about 12 reference analyte spots to about 16 reference analyte spots, about 16 reference analyte spots to about 20 reference analyte spots, about 20 reference analyte spots to about 24 reference analyte spots, about 24 reference analyte spots to about 28 reference analyte spots, about 28 reference analyte spots to about 30 reference analyte spots, about 30 reference analyte spots to about 34 reference analyte spots, about 34 reference analyte spots to about 38 reference analyte spots, about 38 reference analyte spots to about 42 reference analyte spots, about 42 reference analyte spots to about 46 reference analyte spots, about 46 reference analyte spots to about 50 reference analyte spots, about 50 reference analyte spots to about 54 reference analyte spots, about 54 reference analyte spots to about 58 reference analyte spots, about 58 reference analyte spots to about 62 reference analyte spots, about 62 reference analyte spots to about 64 reference analyte spots, or more).

In some embodiments, a reference analyte spot 14048 is spaced away from a second reference analyte spot 14048 by a center-to-center distance of about 0.06 millimeters (mm) to about 1.00 mm (e.g., about 0.06 mm to about 0.08 mm, about 0.08 mm to about 0.10 mm, about 0.10 mm to 0.20 mm, 0.20 mm to about 0.30 mm, about 0.30 mm to about 0.40 mm, about 0.40 mm to about 0.50 mm, about 0.50 mm to about 0.60 mm, about 0.60 mm to about 0.70 mm, about 0.70 mm to about 0.80 mm, about 0.80 mm to about 0.90 mm, about 0.90 mm to about 1.00 mm). In some embodiments, the reference analyte spot 14048 includes a reference analyte at a concentration ranging from about 0.01 micromolar (μM) to about 100 μM or more (e.g., from about 0.01 μM to about 1 μM, about 1 μM to about 10 μM, about 10 μM to about 20 μM, about 20 μM to about 30 μM, about 30 μM to about 40 μM, about 40 μM to about 50 μM, about 50 μM to about 60 μM, about 60 μM to about 70 μM, about 70 μM to about 80 μM, about 80 μM to about 90 μM, about 90 μM to about 100 μM, or more). In some embodiments, the volume of an individual reference analyte spot is ranges from about 10 μL to about 1000 μL, or more (e.g., about 10 μL to about 50 μL, about 50 μL to about 100 μL, about 100 μL to about 150 μL, about 150 μL to about 200 μL, about 200 μL to about 250 μL, about 250 μL to about 300 μL, about 300 μL to about 350 μL, about 350 μL to about 400 μL, about 400 μL to about 450 μL, about 450 μL to about 500 μL, about 500 μL to about 550 μL, about 550 μL to about 600 μL, about 600 μL to about 650 μL, about 650 μL to about 700 μL, about 700 μL to about 750 μL, about 750 μL to about 800 μL, about 800 μL to about 850 μL, about 850 μL to about 900 μL, about 900 μL to about 950 μL, about 950 μL to about 1000 μL, or more).

The reference arrays 14046 arrays can have a specific arrangement of a plurality of features (e.g., reference analyte spot 14048) that is either irregular or forms a regular pattern. Individual features in the reference array can differ from one another based on their relative spatial locations, the length of the reference analyte, the type of fluorescent probe conjugated to the reference analyte, and the concentration of the fluorescent probe and/or reference analyte. The reference arrays 14046 can be fabricated using a variety of techniques including the array fabrication techniques described above and photolithography based techniques. Such techniques include, but are not limited to, “spotting,” printing, stamping, and synthetizing directly onto the substrate. In some embodiments, the arrays are “spotted” or “printed” with oligonucleotides conjugated to a fluorescent probe and these fluorescent labeled-oligonucleotides are then attached to the substrate to form a fluorescent marker. The fluorescent labeled-oligonucleotides can be applied onto the surface of the substrate by either noncontact or contact printing, as described above. In addition to those above, a wide variety of other features can be used to form the arrays described herein. For example, in some embodiments, features that are formed from fluorescent-conjugated polymers and/or biopolymers that are jet printed, screen printed, or electrostatically deposited on the surface of the substrate can be used to form the substrate arrays. In some embodiments, the polymers and biopolymers are fluorescent-conjugated polymers and biopolymers. In some embodiments, a plurality of features can also be synthesized in-situ.

In some embodiments, the substrate includes an array disposed on a surface of the substrate. In some embodiments, the array includes a plurality of reference analyte spots 14048 arranged to form a first pattern. In some embodiments, each reference analyte spot 14048 includes a fluorescent probe.

The reference analyte spots 14048 have the shape of a circular spot, as shown in FIGS. 14A-14B. In some embodiments, the reference analyte spots 14048 have a diameter of about 100 micrometer (m). In some embodiments, the reference analyte spots 14048 comprises an oligonucleotide and a fluorophore. In some embodiments, the fluorophore is attached to a 3′ terminus of the oligonucleotide. In other examples, the fluorophore is attached to a 5′ terminus of the oligonucleotide. In general, reference analyte spots 14048 can have a variety of two-dimensional shapes when viewed in a plane parallel to the substrate. In some embodiments, the reference analyte spots 14048 are provided in the shape of a hexagon, a square, a rectangle, a triangle, a pentagon, a heptagon, an octagon, a nonagon, a decagon, an ellipse, a regular polygon, or any other regular or irregular geometric shape. In some embodiments, the reference analyte spots 14048 have an amorphous shape.

In some embodiments, when a substrate includes more than one reference array, the plurality of reference arrays can include reference analyte spots having substantially the same shape. In some embodiments, some or all of the reference analyte spots of the reference array have an identical or similar shape. In some embodiments, when a substrate includes more than one reference array, the plurality of reference arrays can include reference analyte spots having substantially different shapes and/or one or more shape patterns. In some embodiments, the plurality of reference arrays can include reference analyte spots having 1, 2, 3, 4, 5, 6, 7, 8, 9, 10, or 20 different shape patterns. For example, a reference array can include a first row of reference analyte spots having a first shape (e.g., a circular spot) and a second row of reference analyte spots having a second shape (e.g., a triangular shape) that alternate throughout the substrate array rows. In another example, a reference array can include a repeating unit including a first reference analyte spot having a first shape (e.g., a circular spot), a second reference analyte spot having a second shape (e.g., a triangular shape), and a third reference analyte spot having a third shape (e.g., a square shape) that alternate in a pattern within the same row. In some embodiments, the reference array can include a reference analyte spot pattern having a repeating unit that includes any combinations of different shapes. In some embodiments, some or all of the reference analyte spots of reference arrays can have equal dimensions (e.g., same diameter). In some embodiments, some or all of the reference analyte spots of reference arrays can have different dimensions (e.g., a first diameter that is different than a second diameter).

In some embodiments, the reference analyte spots have a first concentration (or a first average concentration) and a second concentration (or a second average concentration). In other words, in various embodiments, a first reference analyte spot can have first and second concentrations of the reference analyte (e.g., a fluorescent-conjugated oligonucleotide) within the same reference array. In some embodiments, the first concentration is different from the second concentration. In some embodiments, the first concentration ranges from about 0.01 micromolar (μM) to about 100 μM or more (e.g., from about 0.01 μM to about 1 μM, about 1 μM to about 10 μM, about 10 μM to about 20 μM, about 20 μM to about 30 μM, about 30 μM to about 40 μM, about 40 μM to about 50 μM, about 50 μM to about 60 μM, about 60 μM to about 70 μM, about 70 μM to about 80 μM, about 80 μM to about 90 μM, about 90 μM to about 100 μM, or more). In some embodiments, the second concentration ranges from about 0.01 micromolar (μM) to about 100 μM or more (e.g., from about 0.01 μM to about 1 μM, about 1 μM to about 10 μM, about 10 μM to about 20 μM, about 20 μM to about 30 μM, about 30 μM to about 40 μM, about 40 μM to about 50 μM, about 50 μM to about 60 μM, about 60 μM to about 70 μM, about 70 μM to about 80 μM, about 80 μM to about 90 μM, about 90 μM to about 100 μM, or more). In some embodiments, the first concentration is greater than the second concentration. In some embodiments, the first concentration is less than the second concentration.

In some embodiments, the concentrations of the reference analyte spots increase successively (e.g., from about 0.5 μM to about 20 μM). For example, in some embodiments, the reference analyte spots increase concentration starting at a first reference analyte spot of a row and ending at a last reference analyte spot of the same row or vice-versa. In some embodiments, the reference analyte spots increase concentration starting at a first reference analyte spot of a column and ending at a last reference analyte spot of the same column or vice-versa.

In some embodiments, an individual reference analyte spot contains a mixture of fluorescent-labeled oligonucleotides and blank oligonucleotides (e.g., oligonucleotides that are not coupled to a fluorophore at a 5′-terminal end or a 3′ terminal end). In some embodiments, the mixture of fluorescent-labeled oligonucleotides and blank oligonucleotides has a volume of about 50 picoliters (μL).

(ii) Generation of Capture Probes on an Array

Arrays can be prepared by a variety of methods. In some embodiments, arrays are prepared through the synthesis (e.g., in situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical Microbiology Reviews 22.4 (2009): 611-633; US201314111482A; U.S. Pat. No. 9,593,365B2; US2019203275; and WO2018091676, which are each incorporated herein by reference in its entirety.

(1) Spotting or Printing

In some embodiments, oligonucleotides (e.g., capture probes) can be “spotted” or “printed” onto a substrate to form an array. The oligonucleotides can be applied by either noncontact or contact printing. A noncontact printer can use the same method as computer printers (e.g., bubble jet or inkjet) to expel small droplets of probe solution onto the substrate. The specialized inkjet-like printer can expel nanoliter to picoliter volume droplets of oligonucleotide solution, instead of ink, onto the substrate. In contact printing, each print pin directly applies the oligonucleotide solution onto a specific location on the surface. The oligonucleotides can be attached to the substrate surface by the electrostatic interaction of the negative charge of the phosphate backbone of the DNA with a positively charged coating of the substrate surface or by UV-cross-linked covalent bonds between the thymidine bases in the DNA and amine groups on the treated substrate surface. In some embodiments, the substrate is a glass slide. In some embodiments, the oligonucleotides (e.g., capture probes) are attached to a substrate by a covalent bond to a chemical matrix, e.g., epoxy-silane, amino-silane, lysine, polyacrylamide, etc.

(2) In situ Synthesis

Capture probes arrays can be prepared by in situ synthesis. In some embodiments, capture probe arrays can be prepared using photolithography. Photolithography typically relies on UV masking and light-directed combinatorial chemical synthesis on a substrate to selectively synthesize probes directly on the surface of an array, one nucleotide at a time per spot, for many spots simultaneously. In some embodiments, a substrate contains covalent linker molecules that have a protecting group on the free end that can be removed by light. UV light is directed through a photolithographic mask to deprotect and activate selected sites with hydroxyl groups that initiate coupling with incoming protected nucleotides that attach to the activated sites. The mask is designed in such a way that the exposure sites can be selected, and thus specify the coordinates on the array where each nucleotide can be attached. The process can be repeated, a new mask is applied activating different sets of sites and coupling different bases, allowing different oligonucleotides to be constructed at each site. This process can be used to synthesize hundreds of thousands of different oligonucleotides. In some embodiments, maskless array synthesizer technology can be used. Programmable micromirrors can create digital masks that reflect the desired pattern of UV light to deprotect the features.

In some embodiments, the inkjet spotting process can also be used for in situ oligonucleotide synthesis. The different nucleotide precursors plus catalyst can be printed on the substrate and are then combined with coupling and deprotection steps. This method relies on printing picoliter volumes of nucleotides on the array surface in repeated rounds of base-by-base printing that extends the length of the oligonucleotide probes on the array.

(3) Electric Fields

Arrays can also be prepared by active hybridization via electric fields to control nucleic acid transport. Negatively charged nucleic acids can be transported to specific sites, or features, when a positive current is applied to one or more test sites on the array. The surface of the array can contain a binding molecule, e.g., streptavidin, which allows for the formation of bonds (e.g., streptavidin-biotin bonds) once electrically addressed biotinylated probes reach their targeted location. The positive current is then removed from the active features, and new test sites can be activated by the targeted application of a positive current. The process is repeated until all sites on the array are covered.

(4) Ligation

In some embodiments, an array comprising barcoded probes can be generated through ligation of a plurality of oligonucleotides. In some instances, an oligonucleotide of the plurality contains a portion of a barcode, and the complete barcode is generated upon ligation of the plurality of oligonucleotides. For example, a first oligonucleotide containing a first portion of a barcode can be attached to a substrate (e.g., using any of the methods of attaching an oligonucleotide to a substrate described herein), and a second oligonucleotide containing a second portion of the barcode can then be ligated onto the first oligonucleotide to generate a complete barcode. Different combinations of the first, second and any additional portions of a barcode can be used to increase the diversity of the barcodes. In instances where the second oligonucleotide is also attached to the substrate prior to ligation, the first and/or the second oligonucleotide can be attached to the substrate via a surface linker which contains a cleavage site. Upon ligation, the ligated oligonucleotide can be linearized by cleaving at the cleavage site.

To increase the diversity of the barcodes, a plurality of second oligonucleotides comprising two or more different barcode sequences can be ligated onto a plurality of first oligonucleotides that comprise the same barcode sequence, thereby generating two or more different species of barcodes. To achieve selective ligation, a first oligonucleotide attached to a substrate containing a first portion of a barcode can initially be protected with a protective group (e.g., a photocleavable protective group), and the protective group can be removed prior to ligation between the first and second oligonucleotide. In instances where the barcoded probes on an array are generated through ligation of two or more oligonucleotides, a concentration gradient of the oligonucleotides can be applied to a substrate such that different combinations of the oligonucleotides are incorporated into a barcoded probe depending on its location on the substrate.

Probes can be generated by directly ligating additional oligonucleotides onto existing oligonucleotides via a splint oligonucleotide. In some embodiments, oligonucleotides on an existing array can include a recognition sequence that can hybridize with a splint oligonucleotide. The recognition sequence can be at the free 5′ end or the free 3′ end of an oligonucleotide on the existing array. Recognition sequences useful for the methods of the present disclosure may not contain restriction enzyme recognition sites or secondary structures (e.g., hairpins), and may include high contents of Guanine and Cytosine nucleotides.

(5) Polymerases

Barcoded probes on an array can also be generated by adding single nucleotides to existing oligonucleotides on an array, for example, using polymerases that function in a template-independent manner. Single nucleotides can be added to existing oligonucleotides in a concentration gradient, thereby generating probes with varying length, depending on the location of the probes on the array.

(6) Modification of Existing Capture Probes

Arrays can also be prepared by modifying existing arrays, for example, by modifying oligonucleotides already attached to an array. For instance, capture probes can be generated on an array that already comprises oligonucleotides that are attached to the array (or features on the array) at the 3′ end and have a free 5′ end. In some instances, an array is any commercially available array (e.g., any of the arrays available commercially as described herein). The oligonucleotides can be in situ synthesized using any of the in situ synthesis methods described herein. The oligonucleotide can include a barcode and one or more constant sequences. In some instances, the constant sequences are cleavable sequences. The length of the oligonucleotides attached to the substrate (e.g., array) can be less than 100 nucleotides (e.g., less than 90, 80, 75, 70, 60, 50, 45, 40, 35, 30, 25, 20, 15, or 10 nucleotides). To generate probes using oligonucleotides, a primer complementary to a portion of an oligonucleotide (e.g., a constant sequence shared by the oligonucleotides) can hybridize to the oligonucleotide and extend the oligonucleotide (using the oligonucleotide as a template) to form a duplex and to create a 3′ overhang. The 3′ overhang can be created by template-independent ligases (e.g., terminal deoxynucleotidyl transferase (TdT) or poly(A) polymerase). The 3′ overhang allows additional nucleotides or oligonucleotides to be added to the duplex, for example, by an enzyme. For instance, a capture probe can be generated by adding additional oligonucleotides to the end of the 3′ overhang (e.g., via splint oligonucleotide mediated ligation), where the additional oligonucleotides can include a sequence or a portion of sequence of one or more capture domains, or a complement thereof.

The additional oligonucleotide (e.g., a sequence or a portion of sequence of a capture domain) can include a degenerate sequence (e.g., any of the degenerate sequences as described herein). The additional oligonucleotide (e.g., a sequence or a portion of sequence of a capture domain) can include a sequence compatible for hybridizing or ligating with an analyte of interest in a biological sample. An analyte of interest can also be used as a splint oligonucleotide to ligate additional oligonucleotides onto a probe. When using a splint oligonucleotide to assist in the ligation of additional oligonucleotides, an additional oligonucleotide can include a sequence that is complementary to the sequence of the splint oligonucleotide. Ligation of the oligonucleotides can involve the use of an enzyme, such as, but not limited to, a ligase. Non-limiting examples of suitable ligases include Tth DNA ligase, Taq DNA ligase, Thermococcus sp. (strain 9oN) DNA ligase (9oNTM DNA ligase, New England Biolabs), Ampligase™ (available from Lucigen, Middleton, Wis.), and SplintR (available from New England Biolabs, Ipswich, Mass.). An array generated as described above is useful for spatial analysis of a biological sample. For example, the one or more capture domains can be used to hybridize with the poly(A) tail of an mRNA molecule. Reverse transcription can be carried out using a reverse transcriptase to generate cDNA complementary to the captured mRNA. The sequence and location of the captured mRNA can then be determined (e.g., by sequencing the capture probe that contains the barcode as well as the complementary cDNA).

An array for spatial analysis can be generated by various methods as described herein. In some embodiments, the array has a plurality of capture probes comprising spatial barcodes. These spatial barcodes and their relationship to the locations on the array can be determined. In some cases, such information is readily available, because the oligonucleotides are spotted, printed, or synthesized on the array with a pre-determined pattern. In some cases, the spatial barcode can be decoded by methods described herein, e.g., by in situ sequencing, by various labels associated with the spatial barcodes etc. In some embodiments, an array can be used as a template to generate a daughter array. Thus, the spatial barcode can be transferred to the daughter array with a known pattern.

(iii) Substrate Attributes

A substrate can generally have any suitable form or format. For example, a substrate can be flat, curved, e.g., convexly or concavely curved towards the area where the interaction between a biological sample, e.g., tissue sample, and a substrate takes place. In some embodiments, a substrate is flat, e.g., planar, chip, or slide. A substrate can contain one or more patterned surfaces within the substrate (e.g., channels, wells, projections, ridges, divots, etc.).

A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

Substrates can optionally include various structures such as, but not limited to, projections, ridges, and channels. A substrate can be micropatterned to limit lateral diffusion (e.g., to prevent overlap of spatial barcodes). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a substrate can be modified so that discrete sites are formed that can only have or accommodate a single feature. In some embodiments, the surface of a substrate can be modified so that features adhere to random sites.

In some embodiments, the surface of a substrate is modified to contain one or more wells, using techniques such as (but not limited to) stamping, microetching, or molding techniques. In some embodiments in which a substrate includes one or more wells, the substrate can be a concavity slide or cavity slide. For example, wells can be formed by one or more shallow depressions on the surface of the substrate. In some embodiments, where a substrate includes one or more wells, the wells can be formed by attaching a cassette (e.g., a cassette containing one or more chambers) to a surface of the substrate structure.

In some embodiments, the structures of a substrate (e.g., wells or features) can each bear a different capture probe. Different capture probes attached to each structure can be identified according to the locations of the structures in or on the surface of the substrate. Exemplary substrates include arrays in which separate structures are located on the substrate including, for example, those having wells that accommodate features.

In some embodiments where the substrate is modified to contain one or more structures, including but not limited to, wells, projections, ridges, features, or markings, the structures can include physically altered sites. For example, a substrate modified with various structures can include physical properties, including, but not limited to, physical configurations, magnetic or compressive forces, chemically functionalized sites, chemically altered sites, and/or electrostatically altered sites. In some embodiments where the substrate is modified to contain various structures, including but not limited to wells, projections, ridges, features, or markings, the structures are applied in a pattern. Alternatively, the structures can be randomly distributed.

The substrate (e.g., or a bead or a feature on an array) can include tens to hundreds of thousands or millions of individual oligonucleotide molecules (e.g., at least about 10,000, 50,000, 100,000, 500,000, 1,000,000, 10,000,000, 100,000,000, 1,000,000,000, or 10,000,000,000 oligonucleotide molecules).

In some embodiments, a substrate includes one or more markings on a surface of a substrate, e.g., to provide guidance for correlating spatial information with the characterization of the analyte of interest. For example, a substrate can be marked with a grid of lines (e.g., to allow the size of objects seen under magnification to be easily estimated and/or to provide reference areas for counting objects). In some embodiments, fiducial markers can be included on a substrate. Such markings can be made using techniques including, but not limited to, printing, sand-blasting, and depositing on the surface.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, and colorimetric labels. The use of fiducial markers to stabilize and orient biological samples is described, for example, in Carter et al., Applied Optics 46:421-427, 2007), the entire contents of which are incorporated herein by reference. In some embodiments, a fiducial marker can be a physical particle (e.g., a nanoparticle, a microsphere, a nanosphere, a bead, a post, or any of the other exemplary physical particles described herein or known in the art).

In some embodiments, a fiducial marker can be present on a substrate to provide orientation of the biological sample. In some embodiments, a microsphere can be coupled to a substrate to aid in orientation of the biological sample. In some examples, a microsphere coupled to a substrate can produce an optical signal (e.g., fluorescence). In another example, a microsphere can be attached to a portion (e.g., corner) of an array in a specific pattern or design (e.g., hexagonal design) to aid in orientation of a biological sample on an array of features on the substrate. In some embodiments, a quantum dot can be coupled to the substrate to aid in the orientation of the biological sample. In some examples, a quantum dot coupled to a substrate can produce an optical signal.

In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Such a marker nucleic acid molecule can be contacted with an array before, contemporaneously with, or after the tissue sample is stained to visualize or image the tissue section. Although not required, it can be advantageous to use a marker that can be detected using the same conditions (e.g., imaging conditions) used to detect a labelled cDNA.

In some embodiments, fiducial markers are included to facilitate the orientation of a tissue sample or an image thereof in relation to an immobilized capture probes on a substrate. Any number of methods for marking an array can be used such that a marker is detectable only when a tissue section is imaged. For instance, a molecule, e.g., a fluorescent molecule that generates a signal, can be immobilized directly or indirectly on the surface of a substrate. Markers can be provided on a substrate in a pattern (e.g., an edge, one or more rows, one or more lines, etc.).

In some embodiments, a fiducial marker can be randomly placed in the field of view. For example, an oligonucleotide containing a fluorophore can be randomly printed, stamped, synthesized, or attached to a substrate (e.g., a glass slide) at a random position on the substrate. A tissue section can be contacted with the substrate such that the oligonucleotide containing the fluorophore contacts, or is in proximity to, a cell from the tissue section or a component of the cell (e.g., an mRNA or DNA molecule). An image of the substrate and the tissue section can be obtained, and the position of the fluorophore within the tissue section image can be determined (e.g., by reviewing an optical image of the tissue section overlaid with the fluorophore detection). In some embodiments, fiducial markers can be precisely placed in the field of view (e.g., at known locations on a substrate). In this instance, a fiducial marker can be stamped, attached, or synthesized on the substrate and contacted with a biological sample. Typically, an image of the sample and the fiducial marker is taken, and the position of the fiducial marker on the substrate can be confirmed by viewing the image.

In some embodiments, a fiducial marker can be an immobilized molecule (e.g., a physical particle) attached to the substrate. For example, a fiducial marker can be a nanoparticle, e.g., a nanorod, a nanowire, a nanocube, a nanopyramid, or a spherical nanoparticle. In some examples, the nanoparticle can be made of a heavy metal (e.g., gold). In some embodiments, the nanoparticle can be made from diamond. In some embodiments, the fiducial marker can be visible by eye.

As noted herein, any of the fiducial markers described herein (e.g., microspheres, beads, or any of the other physical particles described herein) can be located at a portion (e.g., corner) of an array in a specific pattern or design (e.g., hexagonal design) to aid in orientation of a biological sample on an array of features on the substrate. In some embodiments, the fiducial markers located at a portion (e.g., corner) of an array (e.g., an array on a substrate) can be patterned or designed in at least 1, at least 2, at least 3, or at least 4 unique patterns. In some examples, the fiducial markers located at the corners of the array (e.g., an array on a substrate) can have four unique patterns of fiducial markers.

In some examples, fiducial markers can surround the array. In some embodiments the fiducial markers allow for detection of, e.g., mirroring. In some embodiments, the fiducial markers may completely surround the array. In some embodiments, the fiducial markers may not completely surround the array. In some embodiments, the fiducial markers identify the corners of the array. In some embodiments, one or more fiducial markers identify the center of the array. In some embodiments, the fiducial markers comprise patterned spots, wherein the diameter of one or more patterned spot fiducial markers is approximately 100 micrometers. The diameter of the fiducial markers can be any useful diameter including, but not limited to, 50 micrometers to 500 micrometers in diameter. The fiducial markers may be arranged in such a way that the center of one fiducial marker is between 100 micrometers and 200 micrometers from the center of one or more other fiducial markers surrounding the array. In some embodiments, the array with the surrounding fiducial markers is approximately 8 mm by 8 mm. In some embodiments, the array without the surrounding fiducial markers is smaller than 8 mm by 50 mm.

In some embodiments, an array can be enclosed within a frame. Put another way, the perimeter of an array can have fiducial markers such that the array is enclosed, or substantially enclosed. In some embodiments, the perimeter of an array can be fiducial markers (e.g., any fiducial marker described herein). In some embodiments, the perimeter of an array can be uniform. For example, the fiducial markings can connect, or substantially connect, consecutive corners of an array in such a fashion that the non-corner portion of the array perimeter is the same on all sides (e.g., four sides) of the array. In some embodiments, the fiducial markers attached to the non-corner portions of the perimeter can be pattered or designed to aid in the orientation of the biological sample on the array. In some embodiments, the particles attached to the non-corner portions of the perimeter can be patterned or designed in at least 1, at least 2, at least 3, or at least 4 patterns. In some embodiments, the patterns can have at least 2, at least 3, or at least 4 unique patterns of fiducial markings on the non-corner portion of the array perimeter.

In some embodiments, an array can include at least two fiducial markers (e.g., at least 3, at least 4, at least 5, at least 6, at least 7, at least 8, at least 9, at least 10, at least 12, at least 15, at least 20, at least 30, at least 40, at least 50, at least 60, at least 70, at least 80, at least 90, at least 100 fiducial markers or more (e.g., several hundred, several thousand, or tens of thousands of fiducial markers)) in distinct positions on the surface of a substrate. Fiducial markers can be provided on a substrate in a pattern (e.g., an edge, one or more rows, one or more lines, etc.).

A wide variety of different substrates can be used for the foregoing purposes. In general, a substrate can be any suitable support material. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Among the examples of substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In some embodiments, the biological sample can be placed on any suitable area and surface of the substrate. In some embodiments, any number and configuration (e.g., positioning and/or dimensions) of reference wells, reference arrays, and/or sample regions can be included in the substrates of the disclosure.

(b) Kits

In some embodiments, also provided herein are kits that include one or more substrates and one or more labeled oligonucleotide probes described herein. In some instances, the kit includes a substrate comprising a plurality of capture probes each comprising a spatial barcode and the capture domain. In some instances, the kit includes a substrate comprising a plurality of reservoirs or arrays each comprising a reference analyte of known length and/or fluorescence intensity or FRET efficiency. In some instances, the kit includes a plurality of probes (e.g., a labeled oligonucleotide probes).

A non-limiting example of a kit used to perform any of the methods described herein includes: (a) a substrate comprising a plurality of capture probes wherein a capture probe of the plurality of capture probes comprises a capture domain, (b) a plurality of labeled oligonucleotide probes comprising: (i) a primer oligonucleotide probe comprising a first label, and (ii) a plurality of labeled oligonucleotide probes comprising a second label, wherein the plurality of labeled oligonucleotide probes comprising the second label are substantially complementary to an analyte, or a complement thereof, and (c) instructions for performing any of the methods of the disclosure.

Another non-limiting example of a kit used to perform any of the methods described herein includes (a) a substrate comprising a plurality of capture probes wherein a capture probe of the plurality of capture probes comprises a capture domain, (b) a first labeled oligonucleotide probe and a second labeled oligonucleotide probe, wherein the first labeled oligonucleotide probe is capable of transferring energy to the second labeled oligonucleotide probe by a dipole-dipole coupling mechanism, and (c) instructions for performing any of the methods of the disclosure.

### EXAMPLES

### Example 1—Method of Determining RNA Integrity Via Relative Fluorescence Intensities

This example provides an example method for determining spatial RNA integrity via the detection and comparison of relative fluorescence intensity signals. In a non-limiting example, a formalin fixed, paraffin embedded (FFPE) tissue section sample is prepared prior to being contacted with a substrate of the disclosure. For example, the FFPE tissue section is deparaffinized, imaged, and/or permeabilized by any of the methods described elsewhere herein.

Once the FFPE tissue section is prepared, the FFPE tissue section is contacted with a substrate including a plurality of capture probes. Each capture probe includes a capture domain. Next, 18S rRNA and 28S rRNA molecules from the tissue section are hybridized to the capture probes. The 18S rRNA and 28S rRNA molecules are used as a template to extend a 3′ end of the capture probes generating cDNA molecules from the captured 18S rRNA and 28S rRNA molecules. Next, a plurality of labeled oligonucleotide probes, which are complementary to the 18S rRNA and 28S rRNA molecules, are hybridized to the cDNA molecules. The plurality of labeled oligonucleotide probes complementary to 18S and 28S rRNA molecules includes primer oligonucleotide probes labeled with GFP and Cy3-labeled oligonucleotide probes. Furthermore, a plurality of reference analytes, each having a known length, are deposited in reference wells of the substrate. The reference analytes include primer oligonucleotide probes labeled with GFP and Cy3-labeled oligonucleotides.

Next, the substrate including the reference analytes and the processed FFPE tissue section sample is imaged. Thus, the GFP and Cy3 fluorescence intensity signals of the sample and the reference analytes are detected. Ratios of the Cy3 fluorescence intensity to the GFP fluorescence intensity are calculated for the reference analytes and the sample (e.g., each 18S and 28S rRNA molecule being detected in the sample). The calculated ratios of the sample are compared to the calculated ratios of the reference analytes, and the lengths of the 18S and 28S rRNA molecules in the sample are determined by correlating these ratios to the known lengths of the reference analytes.

A heat map is generated based on the determined lengths of the 18S and 28S rRNA molecules, and the heat map is superimposed on an initial image of the sample (e.g., an image of the sample after being stained with H&E). The heat map indicates a first portion of the sample has longer 18S and/or 28S rRNA molecules than a second portion of the sample. Thus, the first portion of the sample has a higher RNA integrity than the second portion.

### Example 2—Method of Determining RNA Integrity Via FRET

This example provides an example method for determining spatial RNA integrity via detection of the energy transferred between two labeled oligonucleotide probes capable of performing FRET. In a non-limiting example, a frozen tissue section sample is prepared prior to being contacted with a substrate of the disclosure. For example, the frozen tissue section is imaged and/or permeabilized by any of the methods described elsewhere herein.

Once the frozen tissue section is prepared, the frozen tissue section is contacted with a substrate including a plurality of capture probes. Each capture probe includes a capture domain. Next, 18S rRNA and 28S rRNA molecules from the tissue section are hybridized to the capture probes. The 18S rRNA and 28S rRNA molecules are used as a template to extend a 3′ end of the capture probes, thereby generating 18S and 28S cDNA molecules from the captured 18S rRNA and 28S rRNA molecules. Next, a plurality of labeled oligonucleotide probes, which are complementary the 18S and 28S cDNA molecules, are hybridized to key regions of the 18S and 28S cDNA molecules. The labeled oligonucleotide probes complementary to the 18S cDNA molecules are labeled with an “acceptor” fluorescent molecule that is able to perform FRET. Additionally, the labeled oligonucleotide probes complementary to the 28S cDNA molecules are labeled with a “donor” fluorescent molecule that is able to transfer energy to the acceptor fluorescent molecule. Furthermore, a plurality of reference analytes, each having a known length and labeled with acceptor and donor oligonucleotide probes, are deposited in reference wells of the substrate.

Next, the substrate including the reference analytes and the processed frozen tissue section sample is imaged, and the fluorescence intensity signals of the donor and acceptor fluorescent molecules are detected. Next, the FRET efficiencies of the donor and acceptor fluorescent molecules are calculated. The FRET efficiency is defined as the proportion of the donor molecules that have transferred excitation state energy to the acceptor molecules. The oligonucleotide probes labeled with the acceptor and donor molecules are designed to hybridize in series, where the acceptor and donor molecules are arranged in an alternating pattern. A higher FRET efficiency is expected when the 18S and 28S rRNA molecules in the sample are longer due to the increased labeled oligonucleotide probe concentration. Likewise, a lower FRET efficiency is expected when the 18S and 28S rRNA molecules in the sample are shorter due to decreased labeled oligonucleotide probe concentration.

Next, the calculated FRET efficiencies of the sample are compared to the FRET efficiencies of the reference analytes, and the lengths of the 18S and 28S rRNA molecules in the sample are determined by correlating FRET efficiencies ratios to the known lengths of the reference analytes. Next, ratios of the determined length of 28S rRNA to the determined length of 18S rRNA are calculated and averaged. The average ratio is determined to be about 3, thereby indicating the RNA in the sample is of a high quality.

The disclosed compositions and methods can be further understood through the following numbered paragraphs.
1. A method of determining RNA integrity of a biological sample, the method comprising:
   (a) contacting the biological sample with a substrate comprising a plurality of capture probes, wherein a capture probe of the plurality of capture probes comprises a capture domain;
   (b) hybridizing an analyte to the capture probe;
   (c) extending a 3′ end of the capture probe using the analyte as a template to generate an extended capture probe;
   (d) hybridizing a plurality of labeled oligonucleotide probes to the extended capture probe, wherein the plurality of labeled oligonucleotide probes comprises:
      (i) a primer oligonucleotide probe comprising a first label, and
      (ii) a plurality of labeled oligonucleotide probes comprising one or more second labels, wherein the plurality of labeled oligonucleotide probes comprising a second label of the one or more second labels are substantially complementary to the extended capture probe, or a complement thereof;
   (e) detecting a first intensity of the first label and a second intensity of the second label of the plurality of labeled oligonucleotides hybridized to the extended capture probe, thereby detecting a length of the analyte; and
   (f) calculating a ratio of the second intensity to the first intensity, thereby determining the RNA integrity of the biological sample.
2. The method of paragraph 1, wherein the first label comprises a first fluorescent label.
3. The method of paragraph 1 or 2, wherein the second label comprises a second fluorescent label that is different from the first fluorescent label.
4. The method of any one of the preceding paragraphs, further comprising comparing the ratio to a reference, wherein the reference comprises a labeled oligonucleotide having a known length and a known intensity.
5. The method of paragraph 4, wherein the first intensity is indicative of a capture of the analyte.
6. The method of paragraph 4 or 5, wherein the second intensity is indicative of the length of the analyte.
7. The method of any one of the preceding paragraphs, wherein the primer oligonucleotide probe has a length that ranges from about 50 nucleotides to about 100 nucleotides.
8. The method of any one of the preceding paragraphs, wherein each labeled oligonucleotide probe comprising the second label has a length that ranges from about 50 nucleotides to about 100 nucleotides.
9. The method of any one of the preceding paragraphs, wherein each labeled oligonucleotide probe comprising the second label of the plurality of labeled oligonucleotide probes has a length that is about 5% to about 20% of a length of the extended capture probe.
10. The method of any one of the preceding paragraphs, wherein the RNA integrity is high when the ratio of the second intensity to the first intensity is about 3:1 to about 10:1.
11. A method of determining RNA integrity of a biological sample, the method comprising:
   (a) contacting the biological sample with a substrate comprising a plurality of capture probes, wherein a capture probe of the plurality of capture probes comprises a capture domain;
   (b) hybridizing an analyte to the capture probe;
   (c) extending a 3′ end of the capture probe using the analyte as a template to generate an extended capture probe;
   (d) hybridizing a first labeled oligonucleotide probe and a second labeled oligonucleotide probe to the extended capture probe, wherein the first labeled oligonucleotide probe is capable of transferring energy to the second labeled oligonucleotide probe by a dipole-dipole coupling mechanism; and
   (e) determining the energy transferred to the second labeled oligonucleotide probe, thereby detecting a length of the analyte and determining the RNA integrity of the biological sample.
12. The method of paragraph 11, wherein the dipole-dipole coupling mechanism is a Forster resonance energy transfer (FRET).
13. The method of paragraph 11 or 12, wherein the first labeled oligonucleotide probe and the second labeled oligonucleotide probe are hybridized in an alternating pattern to the extended capture probe.
14. The method of any one of paragraphs 11-13, wherein the first labeled oligonucleotide probe and the second labeled oligonucleotide probe have a length that ranges from about 50 nucleotides to about 100 nucleotides.
15. The method of any one of paragraphs 11-14, wherein the first labeled oligonucleotide probe and the second labeled oligonucleotide probe have a length that is about 1% to about 10% of a length of the extended capture probe.
16. The method of any one of paragraphs 12-15, further comprising measuring a FRET efficiency of the first and second labeled oligonucleotide probes.
17. The method of paragraph 16, wherein the RNA integrity is high when the FRET efficiency ranges from about 70% to about 100%.
18. The method of any one of the preceding paragraphs, further comprising generating an image of the extended capture probe and using the image of the extended capture probe to generate a spatial RNA integrity number for a location on the substrate.
19. The method of any one of the preceding paragraphs, wherein the RNA integrity is high when the length of the analyte is about 50% to about 90% of an intact length of the analyte.
20. The method of any one of the preceding paragraphs, wherein the RNA integrity is high when the length of the analyte is about 900 to about 1600 nucleotides.
21. The methods of any one of paragraphs 1-19, wherein the RNA integrity is high when the length of the analyte is about 2500 to about 4500 nucleotides.
22. The method of any one of the preceding paragraphs, wherein an increase in the length of the analyte is correlated to an increase of the RNA integrity of the biological sample.
23. The method of any one of the preceding paragraphs, wherein the biological sample is a tissue sample.
24. The method of paragraph 23, wherein the tissue sample comprises a tissue section, a region within a tissue, or a single cell within a tissue.
25. The method of any one of the preceding paragraphs, wherein the capture domain comprises a sequence complementary to the analyte.
26. The method of any one of paragraphs 1-24, wherein the capture domain comprises a poly(T) sequence.
27. The method of any one of the preceding paragraphs, wherein the analyte is a ribosomal RNA (rRNA).
28. The method of paragraph 27, wherein the rRNA is 18S rRNA, 28S rRNA, or a combination thereof.
29. The method of paragraph 28, further comprising calculating a ratio of the length of the 18S rRNA and/or the 28S rRNA.
30. The method of paragraph 29, wherein the RNA integrity is high when the ratio of the 18S rRNA and/or the 28S rRNA is greater than or equal to about 2.
31. The method of any one of paragraphs 1-26, wherein the analyte is a transfer RNA (tRNA).
32. The method of any one of paragraphs 1-25, wherein the analyte is a highly-expressed analyte.
33. The method of any one of paragraphs 1-31, wherein if the RNA integrity is high, the method further comprises determining abundance and location of a plurality of analytes in a related biological sample, wherein the related biological sample is a serial biological section from the biological sample.
34. The method of paragraph 33, wherein the determining the abundance and location comprises:
   (a) contacting a spatial array comprising a comprising a plurality of spatial capture probes with the serial biological section, wherein a spatial capture probe of the plurality of spatial capture probes comprises a spatial barcode and a spatial capture domain;
   (b) hybridizing an analyte from the serial biological section to a spatial capture domain; and
   (c) determining (i) all or a part of a sequence of the analyte from the serial biological section, or a complement thereof, and (ii) all or a part of a sequence of the spatial barcode, or a complement thereof, and using the determined sequence of (i) and (ii) to determine the abundance and the location of the analyte from the serial biological section.
35. The method of paragraph 34, wherein the determining step comprises amplifying all or part of the analyte from the serial biological section bound to the capture domain, wherein the amplifying creates an amplification product comprising: (i) all or part of the analyte from the serial biological section bound to the capture domain, or a complement thereof, and (ii) all or a part of the sequence of the spatial barcode, or a complement thereof.
36. The method of paragraph 34 or 35, wherein the determining step comprises sequencing.
37. The method of any one of paragraphs 33-36, further comprising imaging the serial biological section.
38. The method of paragraph 34, wherein the spatial capture probe further comprises one or more functional domains, a unique molecular identifier, a cleavage domain, and combinations thereof.
39. A substrate comprising:
   one or more discrete sample regions configured to receive a biological sample;
   a capture probe attached to a location on the sample region, the capture probe configured to hybridize a sample analyte of the biological sample; and
   one or more discrete reservoirs defined by a surface of the substrate and linearly arranged on the substrate, the one or more reservoirs defining a volume configured to receive a fluorescently-labeled reference analyte having a known length.
40. The substrate of paragraphs 39, wherein the fluorescently-labeled reference analyte is 18S rRNA, 28S rRNA, or a combination thereof.
41. The substrate of any one of paragraphs 39 or 40, wherein the known length ranges from about 10 to about 1800 nucleotides.
42. The substrate of any one of paragraphs 39-41, wherein the known length ranges from about 10 to about 5000 nucleotides.
43. The substrate of any one of paragraphs 39-42, wherein the volume ranges from about 20 μL to about 700 μL.
44. A kit comprising:
   (a) a substrate comprising a plurality of capture probes wherein a capture probe of the plurality of capture probes comprises a capture domain;
   (b) plurality of labeled oligonucleotide probes comprising:
      (i) a primer oligonucleotide probe comprising a first fluorescent label, and
      (ii) a plurality of labeled oligonucleotide probes comprising a second fluorescent label, wherein the plurality of labeled oligonucleotide probes comprising the second fluorescent label are substantially complementary to an analyte, or a complement thereof; and
   (c) instructions for performing the methods of any one of paragraphs 1-10 and 18-38.
45. A kit comprising:
   (a) a substrate comprising a plurality of capture probes wherein a capture probe of the plurality of capture probes comprises a capture domain;
   (b) a first labeled oligonucleotide probe and a second labeled oligonucleotide probe, wherein the first labeled oligonucleotide probe is capable of transferring energy to the second labeled oligonucleotide probe by a dipole-dipole coupling mechanism, and wherein the first labeled oligonucleotide comprises a donor fluorophore, and the second labeled oligonucleotide comprises an acceptor fluorophore; and
   (c) instructions for performing the methods of any one of paragraphs 11-38.
46. The kit of paragraphs 44 or 45, wherein the substrate further comprises an array disposed on a surface of the substrate, the array comprising a reference analyte having a known length.
47. The kit of any one of paragraphs 44-46, further comprising a reference analyte having a known length.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining nucleic acid integrity of a biological sample, the method comprising:
(a) contacting the biological sample with a substrate comprising a plurality of capture probes, wherein a capture probe of the plurality of capture probes comprises a capture domain;
(b) hybridizing a nucleic acid analyte of the biological sample to the capture probe;
(c) extending a 3′ end of the capture probe using the nucleic acid analyte as a template to generate an extended capture probe;
(d) hybridizing a plurality of labeled oligonucleotide probes to the extended capture probe, wherein the plurality of labeled oligonucleotide probes comprises:
(i) a primer oligonucleotide probe comprising a first label, and
(ii) a plurality of labeled oligonucleotide probes comprising one or more copies of a second label, wherein the plurality of labeled oligonucleotide probes comprising the one or more copies of the second label are substantially complementary to the extended capture probe, or a complement thereof;
(e) detecting a first intensity of the first label and a second intensity of the second label of the plurality of labeled oligonucleotides hybridized to the extended capture probe, thereby detecting a length of the nucleic acid analyte; and
(f) calculating a ratio of the second intensity to the first intensity, thereby determining the nucleic acid integrity of the biological sample.

2. The method of claim 1, wherein the first label comprises a first fluorescent label.

3. The method of claim 2, wherein the second label comprises a second fluorescent label that is different from the first fluorescent label.

4. The method of claim 1, further comprising comparing the ratio to one or more references, wherein each of the one or more reference comprises a labeled oligonucleotide having a known length and a known intensity.

5. The method of claim 4, wherein the first intensity is indicative of a capture of the nucleic acid analyte by the capture probe.

6. The method of claim 5, wherein the second intensity is indicative of the length of the nucleic acid analyte.

7. The method of claim 1, wherein the primer oligonucleotide probe and/or each labeled oligonucleotide probe comprising the second label has a length that ranges from about 50 nucleotides to about 100 nucleotides.

8. The method of claim 1, wherein each labeled oligonucleotide probe comprising the second label of the plurality of labeled oligonucleotide probes has a length that is about 5% to about 20% of a length of the extended capture probe.

9. The method of claim 1, wherein the nucleic acid integrity is determined to be high when the ratio of the second intensity to the first intensity is about 3:1 to about 10:1.

10. The method of claim 1, further comprising generating an image of the extended capture probe and using the image of the extended capture probe to generate a spatial nucleic acid integrity number for a location on the substrate.

11. The method of claim 1, wherein the nucleic acid analyte is a ribosomal RNA (rRNA) or a transfer RNA (tRNA).

12. The method of claim 11, wherein the rRNA is 18S rRNA, 28S rRNA, or a combination thereof.

13. The method of claim 1, wherein if the nucleic acid integrity is determined to be high, the method further comprises determining abundance and location of a plurality of analytes in a related biological sample, wherein the related biological sample is a serial tissue section from the biological sample.

14. The method of claim 13, further comprising imaging the serial tissue section.

15. The method of claim 13, wherein the determining the abundance and location comprises:
(d) contacting a spatial array comprising a comprising a plurality of spatial capture probes with the serial tissue section, wherein a spatial capture probe of the plurality of spatial capture probes comprises a spatial barcode and a spatial capture domain;
(e) hybridizing an analyte from the plurality of analytes within the serial tissue section to a spatial capture domain; and
(f) determining (i) all or a part of a sequence of the analyte from the serial tissue section, or a complement thereof, and (ii) the spatial barcode, or a complement thereof, and using the determined sequence of (i) and (ii) to determine the abundance and the location of the analyte from the serial tissue section.

16. The method of claim 15, wherein the determining in step (f) comprises amplifying all or part of the analyte hybridized to the capture domain, or a complement thereof, wherein the amplifying creates an amplification product comprising: (i) all or part of the analyte hybridized to the capture domain, or a complement thereof, and (ii) the spatial barcode, or a complement thereof.

17. The method of claim 15, wherein the determining in step (f) comprises sequencing.

18. The method of claim 15, wherein the spatial capture probe further comprises one or more functional domains, a unique molecular identifier, a cleavage domain, or a combination thereof.

19. The method of claim 4, wherein an increase in the length of the nucleic acid analyte compared to a length of a reference of the one or more references correlates to an increase of the nucleic acid integrity of the biological sample.

20. The method of claim 1, wherein the nucleic acid integrity is RNA integrity.

21. The method of claim 1, wherein the biological sample is a tissue sample, wherein the tissue sample is a fresh frozen tissue sample or a fixed tissue sample.

22. The method of claim 1, wherein the capture domain comprises a sequence complementary to the nucleic acid analyte, wherein the nucleic acid analyte hybridizes to the capture probe via the capture domain.

23. A kit comprising:
(a) a substrate comprising a plurality of capture probes wherein a capture probe of the plurality of capture probes comprises a capture domain;
(b) a plurality of labeled oligonucleotide probes comprising:
(i) a primer oligonucleotide probe comprising a first fluorescent label, and
(ii) a plurality of labeled oligonucleotide probes comprising a second fluorescent label, wherein the plurality of labeled oligonucleotide probes comprising the second fluorescent label are substantially complementary to a nucleic acid analyte, or a complement thereof, and
(c) instructions for performing the method of claim 1.

24. A method of determining RNA integrity of a biological sample, the method comprising:
(a) contacting the biological sample with a substrate comprising a plurality of capture probes, wherein a capture probe of the plurality of capture probes comprises a capture domain;
(b) hybridizing an RNA analyte from the biological sample to the capture probe;
(c) extending a 3′ end of the capture probe using the RNA analyte as a template to generate an extended capture probe;
(d) hybridizing a first labeled oligonucleotide probe and a second labeled oligonucleotide probe to the extended capture probe, wherein the first labeled oligonucleotide probe is capable of transferring energy to the second labeled oligonucleotide probe by a dipole-dipole coupling mechanism; and
(e) determining the energy transferred to the second labeled oligonucleotide probe, thereby detecting a length of the RNA analyte and determining the RNA integrity of the biological sample.

25. The method of claim 24, wherein the dipole-dipole coupling mechanism is a Forster resonance energy transfer (FRET),
wherein the method further comprises measuring a FRET efficiency of the first and second labeled oligonucleotide probes.

26. A kit comprising:
(d) a substrate comprising a plurality of capture probes wherein a capture probe of the plurality of capture probes comprises a capture domain;
(e) a first labeled oligonucleotide probe and a second labeled oligonucleotide probe, wherein the first labeled oligonucleotide probe is capable of transferring energy to the second labeled oligonucleotide probe by a dipole-dipole coupling mechanism, and wherein the first labeled oligonucleotide comprises a donor fluorophore, and the second labeled oligonucleotide comprises an acceptor fluorophore; and
(f) instructions for performing the method of claim 24.

27. A substrate comprising:
one or more discrete sample regions configured to receive a biological sample;
a capture probe attached to a location on the one or more discrete sample regions, the capture probe configured to hybridize a sample analyte of the biological sample; and
one or more discrete reservoirs defined by a surface of the substrate and linearly arranged on the substrate, the one or more discrete reservoirs defining a volume configured to receive a fluorescently-labeled reference analyte having a known length.
